SEQUENCE LISTING (26 sequences)

SEQ ID NO: 1 - DNA, 1061 bp, Phytolacca americana
  misc_feature (937)..(937): n is a, c, g, or t
  misc_feature (944)..(944): n is a, c, g, or t
  misc_feature (951)..(951): n is a, c, g, or t
  misc_feature (957)..(957): n is a, c, g, or t
  misc_feature (963)..(963): n is a, c, g, or t
  misc_feature (965)..(965): n is a, c, g, or t
  misc_feature (968)..(968): n is a, c, g, or t
  misc_feature (970)..(970): n is a, c, g, or t
  misc_feature (979)..(979): n is a, c, g, or t
  misc_feature (981)..(981): n is a, c, g, or t
  misc_feature (992)..(992): n is a, c, g, or t
  misc_feature (1022)..(1022): n is a, c, g, or t
  misc_feature (1031)..(1031): n is a, c, g, or t
  misc_feature (1035)..(1035): n is a, c, g, or t
  misc_feature (1042)..(1043): n is a, c, g, or t
  misc_feature (1045)..(1045): n is a, c, g, or t
  misc_feature (1053)..(1053): n is a, c, g, or t
  misc_feature (1056)..(1056): n is a, c, g, or t
  misc_feature (1058)..(1061): n is a, c, g, or t
aaagaagcggcaaagggaagatgaaggtgatgcttgtgcttgtggtgatgataacatcatggctcattcttgcaccgccttcaacttgggccataaatacaatcaccttcgacgctggaaatgcaaccattaacaagtatgctacctttatggaatctcttcgtaatgaagcgaaagatccaagtttaaagtgttatggaataccaatgttgcccaatactaattcaaccatcaagtacttgttggttaagctccaaggtgcaagcctaaaaaccatcacactaatgctaagacgaaacaacttatatgtgatgggctattcggatccctacgacaataagtgtcgttaccatatctttaatgatattaaaggcactgaatacagtgatgtggagaatactctttgcccaagttcaaatcctcgtgttgcaaaacccattaactacaatggcctatatccaactttggaaaaaaaagcaggagtaacctcaagaaatcaagtccaactgggaattcaaatactcagcagtgacattggaaaaatctctggacagggctcgttcactgaaaaaatcgaggccaaattcctgcttgtagccattcaaatggtgtcagaagcagcgcgattcaagtacatagagaatcaggtgaagactaattttaacagagatttctcccctaatgacaaagtacttgacttggaggagaactggggtaagatctctacggcaattcacaattccaagaatggagctttaccaaaacctctagagctaaaaaatgcagacggtactaagtggatagtgcttagagtggatgaaatcaaacctgatgtgggactccttaactatgttaatgggacttgccaagcaacttaacaaaatgccatgttccctgaacttataatgtctacttattataattacatggctaatcntggtganctattcnaaggantctgancntananataatacanantatatatgtantactccaactacattataaaacttaaatanaggccgggncaanggtaccnngntttcttgnacnannnn

SEQ ID NO: 2 - protein, 318 aa, Phytolacca americana
  misc_feature (311)..(311): Xaa can be any naturally occurring amino acid
  misc_feature (313)..(313): Xaa can be any naturally occurring amino acid
  misc_feature (316)..(316): Xaa can be any naturally occurring amino acid
  misc_feature (318)..(318): Xaa can be any naturally occurring amino acid
RSGKGKMKVMLVLVVMITSWLILAPPSTWAINTITFDAGNATINKYATFMESLRNEAKDPSLKCYGIPMLPNTNSTIKYLLVKLQGASLKTITLMLRRNNLYVMGYSDPYDNKCRYHIFNDIKGTEYSDVENTLCPSSNPRVAKPINYNGLYPTLEKKAGVTSRNQVQLGIQILSSDIGKISGQGSFTEKIEAKFLLVAIQMVSEAARFKYIENQVKTNFNRDFSPNDKVLDLEENWGKISTAIHNSKNGALPKPLELKNADGTKWIVLRVDEIKPDVGLLNYVNGTCQATQNAMFPELIMSTYYNYMANXGXLFXGX

SEQ ID NO: 3 - DNA, 2362 bp, Ricinus communis
cctaaaaaaagtaaattactctaatcgacattatatgaattttaactaattccgtttctaatttataattatttcgttaaaccaatcaattccctttaaacactgcttatgcatattctgtctcaatttatatatggcatgcatcttccgtattaatttataagttatttttattgatcaagtatttgtggttttctttatataaaaaaatgtattagtgtttttctgtattaattttataagttcatctttatgagaatgctaatgtatttggacagccaataaaattccagaattgctgcaatcaaagatgaaaccgggaggaaatactattgtaatatggatgtatgcagtggcaacatggctttgttttggatccacctcagggtggtctttcacattagaggataacaacatattccccaaacaatacccaattataaactttaccacagcgggtgccactgtgcaaagctacacaaactttatcagagctgttcgcggtcgtttaacaactggagctgatgtgagacatgaaataccagtgttgccaaacagagttggtttgcctataaaccaacggtttattttagttgaactctcaaatcatgcagagctttctgttacattagcgctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggaggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcaaagacgtaatggttccaaattcagtgtgtacgatgtgagtatattaatccctatcatagctctcatggtgtatagatgcgcacctccaccatcgtcacagttttctttgcttataaggccagtggtaccaaattttaatgctgatgtttgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattattttgatagacagattactctcttgcagtgtgtatgtcctgctatgaaaatagatggcttaaataaaaaggacattgtaaattttgtaactgaaaggacagcaagttattgcagtccagtatctaataagagcacaactattgtcttgtgcattctaaatttatggatgaattgtatgaattaagctaattattttggtcatcagacttgatatctttttgaataaaataaataatatgttttttcaaacttataaatctatgaatgatatgaatataatgcggagactagtcaatcttttatgtaattctatgatgataaaagctt

SEQ ID NO: 4 - protein, 576 aa, Ricinus communis
MKPGGNTIVIWMYAVATWLCFGSTSGWSFTLEDNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFSLLIRPVVPNFNADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 5 - DNA, 23 bp, Artificial Sequence
  Primer
agaagcggcaaagggaagatgaa

SEQ ID NO: 6 - DNA, 22 bp, Artificial Sequence
  Primer
ccattggcccggcctcttattt

SEQ ID NO: 7 - DNA, 780 bp, Phytolacca americana
aatacaatcaccttcgacgctggaaatgcaaccattaacaagtatgctacctttatggaatctcttcgtaatgaagcgaaagatccaagtttaaagtgttatggaataccaatgttgcccaatactaattcaaccatcaagtacttgttggttaagctccaaggtgcaagcctaaaaaccatcacactaatgctaagacgaaacaacttatatgtgatgggctattcggatccctacgacaataagtgtcgttaccatatctttaatgatattaaaggcactgaatacagtgatgtggagaatactctttgcccaagttcaaatcctcgtgttgcaaaacccattaactacaatggcctatatccaactttggaaaaaaaagcaggagtaacctcaagaaatcaagtccaactgggaattcaaatactcagcagtgacattggaaaaatctctggacagggctcgttcactgaaaaaatcgaggccaaattcctgcttgtagccattcaaatggtgtcagaagcagcgcgattcaagtacatagagaatcaggtgaagactaattttaacagagatttctcccctaatgacaaagtacttgacttggaggagaactggggtaagatctctacggcaattcacaattccaagaatggagctttaccaaaacctctagagctaaaaaatgcagacggtactaagtggatagtgcttagagtggatgaaatcaaacctgatgtgggactccttaactatgttaatgggacttgccaagcaact

SEQ ID NO: 8 - DNA, 780 bp, Phytolacca americana
aatacgataacgttcgacgctggaaatgcaaccattaacaagtatgctacctttatggaatctcttcgtaatgaagcgaaagatccaagtttaaagtgttatggaataccaatgttgcccaatactaattcaaccatcaagtacttgttggttaagctccaaggtgcaagcctaaaaaccatcacactaatgctaagacgaaacaacttatatgtgatgggctattcggatccctacgacaataagtgtcgttaccatatctttaatgatattaaaggcactgaatacagtgatgtggagaatactctttgcccaagttcaaatcctcgtgttgcaaaacccattaactacaatggcctatatccaactttggaaaaaaaagcaggagtaacctcaagaaatcaagtccaactaggaattcaaatactcagcagtgacattggaaaaatctctggacagggctcgttcactgaaaaaatcgaggccaaattcctgcttgtagccattcaaatggtgtcagaagcagcgcgattcaagtacatagagaatcaggtgaagactaattttaacagagatttctcccctaatgacaaagtacttgacttggaggagaactggggtaagatctctacggcaattcacaattccaagaatggagctttaccaaaacctctagagctaaaaaatgcagacggtactaagtggatagtgcttagagtggatgaaatcaaacctgatgtgggactccttaactatgttaatggtacctgccaagccact

SEQ ID NO: 9 - DNA, 27 bp, Artificial Sequence
  Primer
gagaatgctaatgtatttggacagcca

SEQ ID NO: 10 - DNA, 25 bp, Artificial Sequence
  Primer
gtattgcgtttccgttgtggaatct

SEQ ID NO: 11 - DNA, 40 bp, Artificial Sequence
  Primer
cagtggtaccaaattttaatataaatacaatcaccttcga

SEQ ID NO: 12 - DNA, 31 bp, Artificial Sequence
  Primer
agaaagctgggtagaacatggcattttgtta

SEQ ID NO: 13 - DNA, 34 bp, Artificial Sequence
  Primer
aaaaagcaggctctataaatacaatcaccttcga

SEQ ID NO: 14 - DNA, 41 bp, Artificial Sequence
  Primer
gtaccactggccttataagcaaagaagttgcttggcaagtc

SEQ ID NO: 15 - DNA, 32 bp, Artificial Sequence
  Primer
aaaaagcaggctctatattccccaaacaatac

SEQ ID NO: 16 - DNA, 40 bp, Artificial Sequence
  Primer
tcgaaggtgattgtatttatattaaaatttggtaccactg

SEQ ID NO: 17 - DNA, 49 bp, Artificial Sequence
  Primer
gcttataaggccagtggtaccaaattttaatatattccccaaacaatac

SEQ ID NO: 18 - DNA, 29 bp, Artificial Sequence
  Primer
gaaagctgggttaaaactgtgacgatggt

SEQ ID NO: 19 - DNA, 32 bp, Artificial Sequence; identical to SEQ ID NO: 15
  Primer

SEQ ID NO: 20 - DNA, 31 bp, Artificial Sequence; identical to SEQ ID NO: 12
  Primer

SEQ ID NO: 21 - DNA, 34 bp, Artificial Sequence; identical to SEQ ID NO: 13
  Primer

SEQ ID NO: 22 - DNA, 29 bp, Artificial Sequence
  Primer
gaaagctgggttaaaactgtgacgatggt

SEQ ID NO: 23 - DNA, 72 bp, Phytolacca americana
atgaaggtgatgcttgtgcttgtggtgatgataacatcatggctcattcttgcaccgccttcaacttgggcc

SEQ ID NO: 24 - DNA, 72 bp, Phytolacca americana
atgaaggtgatgcttgtagttgtggtgacgttaatagcgtggctcattgctgcaccaacttcaacttgtgcc

SEQ ID NO: 25 - protein, 24 aa, Phytolacca americana
MKVMLVLVVMITSWLILAPPSTWA

SEQ ID NO: 26 - protein, 24 aa, Phytolacca americana
MKVMLVVVVTLIAWLIAAPTSTCA

(19) United States
(12) Patent Application Publication
(10) Pub. No.: US 2019/0106466 A1
(43) Pub. Date: Apr. 11, 2019

(54) MEDICINAL PLANTS

(71) Applicant: OPHIUCHUS MEDICINE INC., Vancouver (CA)

(72) Inventor: Yasser Salim HASSAN, Vancouver (CA)

(73) Assignee: OPHIUCHUS MEDICINE INC., Vancouver (CA)

(21) Appl. No.: 16/088,918

(22) PCT Filed: Mar. 28, 2017
(86) PCT No.: PCT/IB2017/000439

§ 371 Date: Sep. 27, 2018

Related U.S. Application Data
(60) Provisional application No. 62/317,679, filed on Apr. 4, 2016.

(51) Int. Cl. C07K 14/415; C12N 15/82; C07K 14/42

(52) U.S. Cl. CPC C07K 14/415; C07K 2319/00; C07K 14/42; C12N 15/8283

(57) ABSTRACT

In one aspect the invention comprises a transgenic garlic plant expressing pokeweed antiviral protein or a fusion protein comprising pokeweed antiviral protein and ricin A-chain. Garlic plants comprising this transgene may possess greater resistance to disease and may provide benefits to animals that consume the plant as antivirals. In another aspect, the invention comprises a vector comprising a polynucleotide that encodes pokeweed antiviral protein or a fusion protein comprising pokeweed antiviral protein and ricin A-chain. In yet another aspect, the invention comprises a fusion protein comprising pokeweed antiviral protein or a fusion protein comprising pokeweed antiviral protein and ricin A-chain. The fusion protein may be delivered to a subject as an extract or in combination with a pharmaceutical excipient as an antiviral agent.

### CROSS REFERENCE TO RELATED APPLICATIONS

This application claims the priority of 62/317,679, filed Apr. 4, 2016, the entire contents of which are hereby incorporated in total by reference.

### FIELD OF THE INVENTION

The present invention generally relates to medicinal plants, means of creating such medicinal plants and uses thereof. More particularly, the present invention relates to a transgenic plant having enhanced defense against plant pathogens and/or enhanced medicinal effect upon consumption; a new signal peptide sequence having roles in toxicity, specificity and expression of Ribosome Inactivating Protein, and a novel fusion protein having roles in numerous therapeutic applications for humans, animals and plants to be used as is or in transgenic plants

### BACKGROUND OF THE INVENTION

Since the industrial revolution and the age of modern technology, man has been changing the environment faster than the environment can adapt. Changes in the way that people live and the conditions in which they work have led to diseases being able to spread much more rapidly, and the emergence of new forms of disease. The rate of mutation of disease is very high, about one per genome per replication for lytic viruses. Due at least in part to these changes, medicinal herbs that were efficient against certain type of diseases are now less efficient or even inefficient against the mutated or new version of the disease. Indeed, plants previously used to treat flu-like symptoms are now inefficient against variants of the influenza virus such as H1N1 and H3N1 subtypes of the virus. Modern medicine is swamped with new diseases everyday that it can barely contain, the latest one being the spread of the Ebola virus, and the cost to society continues to increase. In an effort to prevent disease propagation and minimize related costs, alternative medicines such as Traditional Chinese Medicine (TCM) are receiving increasing attention worldwide. The global demand for herbal medicine is not only large but also growing. What is needed to meet this demand is plants having enhanced medicinal value.

Garlic is a widely used medicinal herb in three of the world's major traditional health systems: Ayurvedic medicine, traditional Chinese medicine and traditional European medicine. It is reputed for having strong medical benefits especially in regards to infections, toxins and poisoning. It is currently the subject of research for many applications ranging from Alzheimer's disease to simple cold prevention. Garlic is also one of the most widely used culinary herbs. China and the United States alone produce 13 million and 170,000 tons per year respectively while India produces 834,000 tons per year. Garlic is widely used for its pungent flavor as a seasoning or condiment, eaten raw or cooked.

Beneficial health properties of garlic may be due to the fact that it includes the organosulfur compound allicin. Allicin is very unstable and quickly changes into a series of other sulfur containing compounds such as diallyl disulfide once garlic is crushed. It exhibits antibacterial, antifungal, antiviral, and antiprotozoal activity and is garlic's defense mechanism against attacks by pests and viruses. However, despite the protection by allicin, garlic today is still very susceptible to plant viruses, this is at least in part because many varieties propagate vegetatively and do not go through a seed stage.

Plants produce toxic proteins known as ribosome inactivating proteins (RIPs) essential to their defense mechanism against outside pathogens. RIPs are RNA N-glycosidases that function by irreversibly inhibiting protein synthesis through the removal of one or more adenine residues from ribosomal RNA (rRNA). In addition, certain RIPs can remove adenine from DNA and other polynucleotides for which reason they are also known as polynucleotide adenosine glycosidases. PAP, a RIP from Phytolacca americana, can cleave not only adenine, but also guanine from the rRNA of Escherichia coli. RIPs are usually categorized into two types, I and II. Type I RIPs are single-chain proteins with a molecular weight of approximately 30 kDa, whereas type II RIPs have an enzymatically active A-chain and a somewhat larger lectin subunit B-chain with a specificity for sugars with galactose-like structures. Type II RIPs have an approximate molecular weight of 56-65 kDa. Given the toxic nature of RIPs, they are usually exported out of the cell once they are synthesized, and are localized either to plant leaves, seeds or roots. Type I RIPs are much more common that type II RIPs and less cytotoxic. This difference in cytotoxicity is believed to be the result of the absence of the cell-binding B chain. However; type I RIPs are still able to enter mammalian cells to some degree in a still poorly understood mechanism. It is hypothesized that they gain access into the cytoplasm as the pathogen enters the cell, thus promoting their activity by impairing host ribosomes. RIPs from plants are being studied as they have potentially useful applications in agriculture and medicine. They have antiviral, antibacterial, antifungal and antitumor activities, which have been exploited in the preparation of immunotoxins (via antibody conjugates) by rendering the activities specifically toxic to the targeted cell. The most active areas in biotechnological research into RIPs is targeted at better understanding and subsequent improvement of the cell entry mechanism, increasing specificity, reducing RIP antigenicity, prolonging their plasma half-life and understanding their role in apoptosis.

Pokeweed, Phytolacca Americana, is a common plant that is often used as a dye. Pokeweed is also eaten when the plant is young and tender, after being boiled. It is used as an antitoxin in both TCM and European traditional medicine. Studies and experiments have shown that a specific protein, the Pokeweed Antiviral Protein (PAP) and its variants, could possibly have a therapeutic use in T-cell leukemia, lymphoma, Hodgkin's lymphoma, and AIDS. For example, the FDA has approved the usage of PAPs by HIV patients in the U.S, and the Medicines Control Council of South Africa has approved the use by African HIV patients. Research is still being conducted to see the effectiveness on other viruses including the common cold. Recent studies have even found that PAPs are efficient against Japanese encephalitis virus. Additionally, PAPs have antiviral activities against many plant viruses. PAPs are believed to be active against a broad spectrum of plant and animal viruses, including poliovirus, herpes simplex, influenza, cytomegalovirus and HIV.

However, it has been observed that some transgenic plants expressing PAPs have phenotype alterations. These alterations may be due to toxicity of PAPs to host cells. Many non-toxic PAPs have been developed to reduce or completely get rid of the toxicity of PAPs to the host cells. However, those PAPs variants have different antiviral activities than wild type PAPs. Wild type PAPs have never been transfected into transgenic garlic.

Pokeweed antiviral protein (PAP) is a potent type I RIP expressed in several organs of the plant pokeweed (Phytolacca americana), secreted and bound within the plant cell wall matrix. It attacks both prokaryotic and eukaryotic ribosomes and, thus, inhibits protein synthesis. Among the PAP gene family, different genes are expressed in various tissues and at different stages of development in Phytolacca americana. PAP, PAP II, PAP-S1, PAP-S2 and PAP-R are the forms that appear in spring leaves, summer leaves, isoform 1 and 2 in seeds, and roots, respectively. The molecular weight ranges from 29 kDa for PAP to 30 kDa for PAP-S. PAP-S1 has been identified as the most effective in inhibiting protein synthesis in vitro among the isoforms produced in seeds. PAPs possess antiviral activity on a wide range of plant and human viruses. PAPs were found to inhibit infection of HEp-2 cells by herpes simplex virus and poliovirus and also the replication of human immunodeficiency virus 1 (HIV-1) in isolated mononuclear blood cells infected in vitro. It was also found that PAPs were more effective than other RIPs at inhibiting the expression of reverse transcriptase in infected cells. It was also found in a recent study that PAPs were efficient against Japanese encephalitis virus. It was also observed that expression of PAPs in transgenic plants leads to broad-spectrum resistance to viral and fungal infections. PAPs have moderate cytotoxicity to non-infected cells and, thus, offer unique opportunities for new applications in therapy and as protective proteins against pathogens in transgenic plants.

Ricin, one of the most potent type II RIPs, produced in the seeds of the castor oil plant, Ricinus communis, can efficiently deliver it's A chain into the cytosol of intoxicated cells through the action of its B chain. The B chain serves as galactose/N-acetylgalactosamine binding domain (lectin) and is linked to the A chain via disulfide bonds. After ricin B chain binds complex carbohydrates on the surface of eukaryotic cells containing either terminal N-acetylgalactosamine or beta-1,4-linked galactose residues, it is endocytosed via clathrin-dependent as well as clathrin-independent endocytosis and is thereafter delivered into the early endosomes. It is then transported to the Golgi apparatus by retrograde transport to reach the endoplasmic reticulum (ER) where its disulfide bonds are cleaved by thioredoxin reductases and disulfide isomerases. The median lethal dose (LD₅₀) of ricin is around 22 micrograms per kilogram of body weight if the exposure is from injection or inhalation (1.78 milligram for an average adult). It is important to note that the ricin A chain on its own has less than 0.01% of the toxicity of the native lectin in a cell culture test system. There are no commercially available therapeutic applications of ricin. While Ricin A chain is used in the development of immunotoxins, it has been shown that ricin A chain alone had no activity on non-infected and tobacco mosaic virus (TMV)-infected tobacco protoplasts alike, while PAPs caused a complete inhibition of TMV production in the infected cells while having no activity on the uninfected protoplasts. Those results led to the conclusion that PAPs, which normally have no mechanism to enter the protoplast, gain entrance to the cytosol of infected cells. It is important to note that in a cell free protein synthesis inhibition assay, it was found that ricin A chain was much more potent than PAPs on rat ribosomes inactivation (in the order of 85 fold stronger). The exact mechanism of the internalization of type 1 RIPs is still unknown, but this attribute is lacking in the A chain of type II RIPs.

One of the current strategies to develop newer antiviral therapeutics is by creating fusion proteins with higher specificity and selectivity in their interactions leading to less side effects and greater potencies than the proteins on their own. One such example was achieved by Rothan et al. where a peptide-fusion recombinant protein LATA-PAP1-THAN (Latarcin-Pokeweed Antiviral Protein I-Thanatin) was found to inhibit Chikungunya virus (CHIKV) replication in the Vero cells at an EC₅₀ of 11.21 g/ml, which is approximately half of the EC50 of PAP1 (23.71 g/ml) and protected the CHIKV-infected mice at the dose of 0.75 mg/ml.

Given the very particular properties of PAP and RTA, those proteins have been extensively studied for the past thirty years for many applications. However, researchers were never able to harness their potentials in an effective way, i.e. without side effects. Indeed, it was found that while those proteins had a very low cytotoxicity to non-infected cells, PAP's administration in a mouse model resulted in hepatic, renal and gastrointestinal tract damage with an LD₅₀ as low as 1.6 mg/Kg. RTA on the other hand showed no toxicity even at the highest dose with similar half-life time. However, all RIP's showed immunosuppressive effects to various degrees. They also showed various side effects when used as immunotoxins (i.e. vascular leak syndrome, hemolytic uremic syndrome, and pluritis among others). Most studies showed that those side effects are the only dose-limiting factors and yet, even at dosages below the toxicity level, in some cases, most of the patients achieved complete or partial remission against Refractory B-Lineage Acute Lymphoblastic Leukemia.

What is needed is an improved garlic plant that would have enhanced resistance to plant viruses and would have improved benefit to humans upon consumption. What is further needed is a new signal peptide sequence having enhanced roles in toxicity, specificity and expression of Ribosome Inactivating Protein (RIP). What is also needed are novel fusion proteins having enhanced therapeutic properties, fewer side effects and less cytoxicity to healthy cells than PAP alone.

### SUMMARY OF THE INVENTION

The following presents a simplified summary in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

The present invention provides a transgenic plant expressing Pokeweed Antiviral Protein (PAP) and/or a novel fusion protein comprising PAP and a type II RIP. In some embodiments, the plant is a member of genus Allium, in other embodiments the plant is Allium sativum. In some embodiments the plant is a garlic plant and in further embodiments the plant is a purple stripe garlic plant. In various embodiments the plant may be a medicinal plant. In some embodiments the fusion protein comprises Ricin A Chain (RTA) and PAP.

It is an object of the invention to enhance general health in a fashion similar to how plant biotechnology presently combats malnutrition through the engineering of nutritionally enhanced crops

It is a further object of the invention to provide a garlic plant having enhanced resistance to plant viruses.

It is a further object of the invention to provide a garlic plant having improved benefit to humans upon consumption as compared to traditional garlic, particularly as related to antiviral properties.

It is a further object of the invention to provide a garlic plant having improved antiviral properties to humans upon consumption as compared to traditional garlic.

It is a further object of the invention to provide a transgenic garlic plant where the expressed pokeweed antiviral proteins are mediated by Agrobacterium tumefaciens transformation.

It is a further object of the invention to provide a transgenic garlic plant wherein the expression of PAP and/or a novel fusion protein does not interfere with garlic growth.

It is a further object of the invention to provide a garlic plant having improved antiviral benefit to animals upon consumption as compared to traditional garlic.

It is a further object of the invention to provide a new signal peptide sequence, which is indicated by the underlined sequence in bold while the mature peptide sequence is indicated by the non-underlined sequence in bold, having new roles in toxicity, specificity and expression of (Ribosome Inactivating Protein) RIP when associated to RIP or any other type of protein.

It is a further object of the invention to provide novel fusion proteins having enhanced antiviral properties and less cytoxicity to healthy cells than PAP alone. The fusion proteins having antiviral properties may be expressed in a garlic plant or any other type of organism or as a standalone or combined therapeutic.

It is a further object of the invention to provide novel fusion proteins that are constructed with a type I RIP, with or without the signal peptide, and the A chain of a type II RIP, with or without the natural polylinker of the type II RIP linking the A chain to the B Chain. The novel fusion protein in this particular case is created by joining the two genes coding for RTA and PAPs respectively, with or without the signal peptide and natural polylinker. Translation of this fusion gene results in a protein or proteins with functional properties derived from each of the original proteins

It is a further object of the invention to provide novel fusion proteins as above with conjugates where the conjugate has the structure: X—Y—Z, wherein X is full length (recombinant fusion protein between RTA and PAPs) rRTA/PAP, containing the new signal peptide or not, having an N-terminal cysteine or not; Y is absent or a chemical linker or a polylinker, and Z is a compound consisting of: an antibody; a hormone; a modified hormone releasing factor; and a hormone releasing factor.

It is a further object of the invention to provide novel fusion proteins that are conjugates of RTA/PAP where the conjugate has the structure: X—Y—Z, wherein X is a compound consisting of: an antibody; a hormone; a modified hormone releasing factor; and a hormone releasing factor, having a N-terminal cysteine or not, containing or not the new signal peptide; Y is absent or a chemical linker or a polylinker, and Z is full length rRTA/PAP containing or not the signal peptide.

### BRIEF DESCRIPTION OF THE DRAWINGS

For a more complete understanding of the nature and desired objects of the present invention, reference is made to the following detailed description taken in conjunction with the accompanying drawing figures wherein like reference characters denote corresponding parts throughout the several views.

FIG. 1 illustrates the pK7WG2D vector.

FIG. 2 illustrates pPAP-HPT-GFP vectors.

FIG. 3 illustrates DNA structures for the expression vectors of (top) PAP-S1 and (middle) Ricin-A-chain/PAP-S1 fusion protein. The expression vector shown at the bottom of FIG. 3 includes a PAP-S1/Ricin-A-chain, T7 promoter (T7), Ribosome binding site (RBS), ATG Start codon, Polyhistidine (6×His) region, Xpress™ epitope, Enterokinase (EK) recognition sit, attR1 site. The expression vector shown at the top of FIG. 3 has a PAP-S1 insert without signal peptide, attR2 site, T7 transcription termination region. The expression vector shown at the middle of FIG. 3 has a Ricin A-chain insert without the signal peptide but with the linker peptide region at the C terminus, PAP-S1 insert with the ricin-A-chain linker peptide region at the N terminus. At the bottom of FIG. 3, the expression vector shown has a Ricin A-chain insert without the signal peptide but with the linker peptide region at the N terminus, PAP-S1 insert with the ricin-A-chain linker peptide region at the C terminus.

FIG. 4 illustrates the T-DNA structure of the binary vector pH7WG2D constructed for plant expression mediated by A. tumefaciens transformation. Arrows indicate transcription orientation. LB is the left border; P35S and T35S, CaMV35S promoter and terminator; Hyg is the Hygromycin resistance gene, Sm/SpR is the Spectinomycin resistance gene, (top) PAP-S1 is the insert with its signal peptide, (bottom) Ricin-A . . . PAP-S1 is the insert with the PAP-S1 signal peptide at the N terminus of Ricin-A-Chain and the linker peptide region at the C terminus of Ricin-A-chain and N terminus of PAP-S1.

FIG. 5 shows recombinant proteins produced in a study described herein. Shown in Lane 1 is the Control protein after His-Tag Purification. The band is clearly visible at 17 kDa. Lane 2 shows PAP-S1 recombinant protein barely visible at the 32 kDa line. Lane 3 shows F1 (at the PAP-S1 C terminus) recombinant protein clearly visible at the 60 kDa line Lane 4 shows F2 (at the PAP-S1 N terminus) recombinant protein visible at the 60 kDa line, but less pronounced than the F1 band.

FIG. 6 shows results of protein activity assays in a study described herein. Lane 1 shows the Control (blur at 17 kDa); shown in Lane 3 is PAP-S1 36% volume of reaction (50 μl) (18 μl of His-Tag purified PAP-S1); shown in Lane 4 is F1 36% volume (18 μl of His-Tag purified F1); and, shown in Lane 5 is F2 36% volume (18 μl of His-Tag purified F2).

FIG. 7 shows results of protein activity assays in a study described herein. Lane 2 shows Control DNA at 17 kDa; shown in Lane 3 is PAP-S1 20% volume of reaction (50 μl) (10 μl of His-Tag purified PAP-S1); shown in Lane 4 is F1 20% volume (10 μl of His-Tag purified F1); and, shown in Lane 5 is F2 20% volume (10 μl of His-Tag purified F2)

FIG. 8 shows results of protein activity assays in a study described herein. Lane 1 shows Control DNA at 17 kDa; shown in Lane 3 is PAP-S1 11.5% volume of reaction (50 μl reaction+50 ul buffer) (5.75 μl of His-Tag purified PAP-S1); shown in Lane 4 is F1 11.5% volume (5.75 μl of His-Tag purified F1); shown in Lane 5 is F2 11.5% volume (5.75 μl of His-Tag purified F2); Lane 6: F2 25% volume (15 μl of His-Tag purified F2); shown in Lane 7 is F1 36% volume (25 μl of His-Tag purified F1); and, shown in Lane 8 is F2 36% volume (25 μl of His-Tag purified F2).

FIG. 9 is a gel showing recombinant proteins gel stained with Coomassie blue after His-Tag purification. Lane 1: Control protein after His-Tag Purification. The band is clearly visible at 17 kDa. Lane 2: PAP-S1 recombinant protein at the 32 k Da line. Lane 3: PAP-S1/RTA recombinant protein at the 60 kDa line. Lane 4: RTA/PAP-S1 recombinant protein at the 60 kDa line. All other bands are due to proteins going through the His-Tag purification column from the initial expression reaction.

FIG. 10 is a graph depicting the activity of recombinant proteins in E. coli protein synthesis. The Y-axis represents percent inhibition compared to control and the X-axis represents the concentration of each respective protein in nM.

FIG. 11A is an image of a stained gel of RTA/PAP-S1R68G from third wash (sample W) and from pooled eluted fraction (sample E) at 60 kDa (top arrow) and of PAP-S1R68G at 30 kDa (bottom arrow), all from native environment, after buffer exchange. The purity of sample W was 65%, of sample E 55% and of PAP-S1R68G 60%

FIG. 11B is close up image of gels and a Western Blot of sample W and E. A double band is showing probably due to degradation of the protein or as a result of an unwanted recombination by E. coli cells.

FIG. 12 is a graph depicting the activity of recombinant proteins in rabbit reticulate lysate in a TnT® system. The Y-axis represents percent inhibition compared to control and the X-axis represents the concentration of each respective protein in nM. Results represent the average for two individual experiments.

FIG. 13 is a graph depicting the bioactivity of proteins recovered from the flow through. The Y-axis represents percent inhibition compared to control and the X-axis represents the concentration of each respective protein in nM. Results represent the average for two individual experiments.

### DETAILED DESCRIPTION

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, analytical chemistry, organic chemistry, and nucleic acid chemistry and hybridization are those well-known and commonly employed in the art.

Standard techniques or modifications thereof are used for chemical syntheses and chemical analyses.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

Standard techniques are used for nucleic acid and peptide synthesis. The techniques and procedures are generally performed according to conventional methods in the art and various general references (e.g., Sambrook & Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

As used herein, the term “RIP” refers to ribosome inactivating proteins.

As used herein, the terms “PAP” or “pokeweed antiviral protein” refer to a polypeptide with substantial or complete sequence homology to pokeweed antiviral protein or a polynucleotide encoding such a polypeptide, which may or may not include a signal peptide as evident by the context in which the term is used (for example, GenBank Entry Accession No. KT630652). When no variant is specified, PAP may refer to the unmodified polypeptide or polynucleotide or to a variant of PAP, by way of non-limiting example, PAP-S1 or PAP-X. As an illustrative and non-limiting example, below is a complete nucleotide sequence of PAP-S1. The signal peptide sequence is indicated by the underlined sequence in bold. The mature peptide sequence is indicated by the non-underlined sequence in bold.

Final Sequence:
SEQ ID NO: 1
AAAGAAGCGGCAAAGGGAAGATGAAGGTGATGCTTGTGCTTGTGGTGATG
ATAACATCATGGCTCATTCTTGCACCGCCTTCAACTTGGGCCATAAATAC
AATCACCTTCGACGCTGGAAATGCAACCATTAACAAGTATGCTACCTTTA
TGGAATCTCTTCGTAATGAAGCGAAAGATCCAAGTTTAAAGTGTTATGGA
ATACCAATGTTGCCCAATACTAATTCAACCATCAAGTACTTGTTGGTTAA
GCTCCAAGGTGCAAGCCTAAAAACCATCACACTAATGCTAAGACGAAACA
ACTTATATGTGATGGGCTATTCGGATCCCTACGACAATAAGTGTCGTTAC
CATATCTTTAATGATATTAAAGGCACTGAATACAGTGATGTGGAGAATAC
TCTTTGCCCAAGTTCAAATCCTCGTGTTGCAAAACCCATTAACTACAATG
GCCTATATCCAACTTTGGAAAAAAAAGCAGGAGTAACCTCAAGAAATCAA
GTCCAACTGGGAATTCAAATACTCAGCAGTGACATTGGAAAAATCTCTGG
ACAGGGCTCGTTCACTGAAAAAATCGAGGCCAAATTCCTGCTTGTAGCCA
TTCAAATGGTGTCAGAAGCAGCGCGATTCAAGTACATAGAGAATCAGGTG
AAGACTAATTTTAACAGAGATTTCTCCCCTAATGACAAAGTACTTGACTT
GGAGGAGAACTGGGGTAAGATCTCTACGGCAATTCACAATTCCAAGAATG
GAGCTTTACCAAAACCTCTAGAGCTAAAAAATGCAGACGGTACTAAGTGG
ATAGTGCTTAGAGTGGATGAAATCAAACCTGATGTGGGACTCCTTAACTA
TGTTAATGGGACTTGCCAAGCAACTTAACAAAATGCCATGTTCCCTGAAC
TTATAATGTCTACTTATTATAATTACATGGCTAATCNTGGTGANCTATTC
NAAGGANTCTGANCNTANANATAATACANANTATATATGTANTACTCCAA
CTACATTATAAAACTTAAATANAGGCCGGGNCAANGGTACCNNGNTTTCT
TGNACNANNNN

As a further non-limiting example, below is the protein sequence translation of the nucleotide sequence of PAP-S1 shown above. The signal peptide sequence is indicated by the underlined sequence in bold. The mature peptide sequence is indicated by the non-underlined sequence in bold.

SEQ ID NO: 2
R S G K G K M K V M L V L V V M I T S W L I L A P
P S T W A I N T I T F D A G N A T I N K Y A T F M
E S L R N E A K D P S L K C Y G I P M L P N T N S
T I K Y L L V K L Q G A S L K T I T L M L R R N N
L Y V M G Y S D P Y D N K C R Y H I F N D I K G T
E Y S D V E N T L C P S S N P R V A K P I N Y N G
L Y P T L E K K A G V T S R N Q V Q L G I Q I L S
S D I G K I S G Q G S F T E K I E A K F L L V A I
Q M V S E A A R F K Y I E N Q V K T N F N R D F S
P N D K V L D L E E N W G K I S T A I H N S K N G
A L P K P L E L K N A D G T K W I V L R V D E I K
D V G L L N Y V N G T C Q A T Stop Q N A M F P E
L I M S T Y Y N Y M A N X G X L F X G X Stop

As used herein, the terms “RTA” or “ricin A-chain” refer to a polypeptide or a polynucleotide encoding a polypeptide with substantial or complete sequence homology to ricin A-chain GenBank Entry Accession No. X52908.1, below.

| SEQ ID NO: 3 | |
|---|---|
| 1 | cctaaaaaaa gtaaattact ctaatcgaca ttatatgaat tttaactaat tccgtttcta |
| 61 | atttataatt atttcgttaa accaatcaat tccctttaaa cactgcttat gcatattctg |
| 121 | tctcaattta tatatggcat gcatcttccg tattaattta taagttattt ttattgatca |
| 181 | agtatttgtg gttttcttta tataaaaaaa tgtattagtg tttttctgta ttaattttat |
| 241 | aagttcatct ttatgagaat gctaatgtat ttggacagcc aataaaattc cagaattgct |
| 301 | gcaatcaaag atgaaaccgg gaggaaatac tattgtaata tggatgtatg cagtggcaac |
| 361 | atggctttgt tttggatcca cctcagggtg gtctttcaca ttagaggata acaacatatt |
| 421 | ccccaaacaa tacccaatta taaactttac cacagcgggt gccactgtgc aaagctacac |
| 481 | aaactttatc agagctgttc gcggtcgttt aacaactgga gctgatgtga gacatgaaat |
| 541 | accagtgttg ccaaacagag ttggtttgcc tataaaccaa cggtttattt tagttgaact |
| 601 | ctcaaatcat gcagagcttt ctgttacatt agcgctggat gtcaccaatg catatgtggt |
| 661 | cggctaccgt gctggaaata gcgcatattt ctttcatcct gacaatcagg aagatgcaga |
| 721 | agcaatcact catcttttca ctgatgttca aaatcgatat acattcgcct ttggaggtaa |
| 781 | ttatgataga cttgaacaac ttgctggtaa tctgagagaa aatatcgagt tgggaaatgg |
| 841 | tccactagag gaggctatct cagcgcttta ttattacagt actggtggca ctcagcttcc |
| 901 | aactctggct cgttccttta taatttgcat ccaaatgatt tcagaagcag caagattcca |
| 961 | atatattgag ggagaaatgc gcacgagaat taggtacaac cggagatctg caccagatcc |
| 1021 | tagcgtaatt acacttgaga atagttgggg gagactttcc actgcaattc aagagtctaa |
| 1081 | ccaaggagcc tttgctagtc caattcaact gcaaagacgt aatggttcca aattcagtgt |
| 1141 | gtacgatgtg agtatattaa tccctatcat agctctcatg gtgtatagat gcgcacctcc |
| 1201 | accatcgtca cagttttctt tgcttataag gccagtggta ccaaatttta atgctgatgt |
| 1261 | ttgtatggat cctgagccca tagtgcgtat cgtaggtcga aatggtctat gtgttgatgt |
| 1321 | tagggatgga agattccaca acggaaacgc aatacagttg tggccatgca agtctaatac |
| 1381 | agatgcaaat cagctctgga ctttgaaaag agacaatact attcgatcta atggaaagtg |
| 1441 | tttaactact tacgggtaca gtccgggagt ctatgtgatg atctatgatt gcaatactgc |
| 1501 | tgcaactgat gccacccgct ggcaaatatg ggataatgga accatcataa atcccagatc |
| 1561 | tagtctagtt ttagcagcga catcagggaa cagtggtacc acacttacag tgcaaaccaa |
| 1621 | catttatgcc gttagtcaag gttggcttcc tactaataat acacaacctt ttgtgacaac |
| 1681 | cattgttggg ctatatggtc tgtgcttgca agcaaatagt ggacaagtat ggatagagga |
| 1741 | ctgtagcagt gaaaaggctg aacaacagtg ggctctttat gcagatggtt caatacgtcc |
| 1801 | tcagcaaaac cgagataatt gccttacaag tgattctaat atacgggaaa cagttgtcaa |
| 1861 | gatcctctct tgtggccctg catcctctgg ccaacgatgg atgttcaaga atgatggaac |
| 1921 | cattttaaat ttgtatagtg ggttggtgtt agatgtgagg gcatcggatc cgagccttaa |
| 1981 | acaaatcatt ctttaccctc tccatggtga cccaaaccaa atatggttac cattattttg |
| 2041 | atagacagat tactctcttg cagtgtgtat gtcctgctat gaaaatagat ggcttaaata |
| 2101 | aaaaggacat tgtaaatttt gtaactgaaa ggacagcaag ttattgcagt ccagtatcta |
| 2161 | ataagagcac aactattgtc ttgtgcattc taaatttatg gatgaattgt atgaattaag |
| 2221 | ctaattattt tggtcatcag acttgatatc tttttgaata aaataaataa tatgtttttt |
| 2281 | caaacttata aatctatgaa tgatatgaat ataatgcgga gactagtcaa tcttttatgt |
| 2341 | aattctatga tgataaaagc tt |

SEQ ID NO: 4
MKPGGNTIVIWMYAVATWLCFGSTSGWSFTLEDNNIFPKQYIPPINFTTAG
ATVQSYTNFIRAVRGRLTTGADVRHEIPVLPNRVGLPINQRFILVELSNHA
ELSVTLALDVTNAYVVGYRAGNSAYFFHPDNQEDAEAITHLFTDVQNRY
TFAFGGNYDRLEQLAGNLRENIELGNGPLEEAISALYYYSTFFTQLPTLAR
SFIICIQMISEAARFQYIEGEMRTRIRYNRRSAPDPSVITLENSWGRLSTAIQ
ESNQGAFASPIQLQRRNGSKFSVYDVSILPIIALMVYRCAPPPSSQFSLLIR
PVVPNFNADVCMDPEPIVRIVGRNGLCVDVRDGRFHNGNAIQLWPCKSN
TDANQLWTLKRDNTIRSNGKCLTTYGYSPGVYVMIYDCNTAATDATRW
QIWDNGTIINPRSSLVLAATSGNSGTTLTVQTNIYAVSQGWLPTNNTQPFV
TTIVGLYGLCLQANSGQVWIEDCSSEKAEQQWALYADGSIRPQQNRDNC
LTSDSNIRETVVKILSCGPASSGQRWMFKNDGTILNLYSGLVLDVRASDPS
LKQIILYPLHGDPNQIWLPLF

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein “endogenous” refers to any material from or produced inside an organism, cell, tissue or system.

As used herein, the term “exogenous” refers to any material introduced from or produced outside an organism, cell, tissue or system.

An “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides, and particularly at the carboxy- or amino-terminus, can be modified by methylation, amidation, acetylation or substitution with other chemical groups which can change a peptide's circulating half-life without adversely affecting activity of the peptide. Additionally, a disulfide linkage may be present or absent in the peptides.

As used herein, the terms “protein”, “peptide” and “polypeptide” are used interchangeably, and refer to a compound comprised of amino acid residues covalently linked by peptide bonds. The term “peptide bond” means a covalent amide linkage formed by loss of a molecule of water between the carboxyl group of one amino acid and the amino group of a second amino acid. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Proteins” include, for example, biologically active fragments, substantially homologous proteins, oligopeptides, homodimers, heterodimers, variants of proteins, modified proteins, derivatives, analogs, and fusion proteins, among others. The proteins include natural proteins, recombinant proteins, synthetic proteins, or a combination thereof. A protein may be a receptor or a non-receptor. In various embodiments, the polypeptides of the invention may have 100% sequence identity to the sequences presented herein, listed in the corresponding entry in Genbank or other databases consulted by people of skill in the art, other embodiments may have 95% or greater sequence identity or 98% or greater sequence identity.

The term “recombinant polypeptide” as used herein is defined as a polypeptide produced by using recombinant DNA methods.

The term “chemical linker” as used herein is defined as a flexible linker, within some embodiments, the linker is a heterobifunctional linker, in some embodiments, the linker comprises a maleimido group. In various embodiments, the linker is selected from the group consisting of: GMBS; EMCS; SMPH; SPDP; and LC-SPDP.

The term “polylinker” or “linker peptide” as used herein is defined as a short segment of DNA added between the DNA encoding the fused proteins, to produce a short peptide or polypeptide to make it more likely that the proteins fold independently and behave as expected. This “polylinker” or “linker peptide” can also have cleavage sites for proteases or chemical agents that enable the liberation of the two separate proteins,

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks the biological activity of the antigen.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

The term “DNA” as used herein is defined as deoxyribonucleic acid.

The term “RNA” as used herein is defined as ribonucleic acid.

The term “recombinant DNA” as used herein is defined as DNA produced by joining pieces of DNA from different sources.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. In various embodiments, the polynucleotides of the invention may have 100% sequence identity to the sequences presented herein, listed in the corresponding entry in Genbank or other databases consulted by people of skill in the art, other embodiments may have 98% sequence identity or 95% sequence identity.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “heterologous” as used herein is defined as DNA or RNA sequences or proteins that are derived from the different species.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 3′ATTGCC5′ and 3′TATGGC are 50% homologous.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for plants include the use of gold nanoparticles and the use of a viral vector such as Agrobacterium tumefaciens. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

As used herein, the term “transgenic” as applied to an organism means that the organism expresses at least one heterologous gene.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like.

By the term “specifically binds,” as used herein, is meant a molecule, such as an antibody, which recognizes and binds to another molecule or feature, but does not substantially recognize or bind other molecules or features in a sample.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

One embodiment of the present invention is a transgenic garlic expressing Pokeweed Antiviral Protein (PAP) and/or a novel fusion protein constructed between RTA and PAPs (RTA/PAP). It is presently contemplated that transgenic garlic expressing Pokeweed PAPs and/or RTA/PAP (altered or native) will allow the garlic herb to not only be more resistant to plant viruses but to also benefit greatly its antiviral properties when consumed, rendering it the herb and food of choice in infection prevention and treatment in herbal medicine. This synergistic effect will be due at least in part to the combination of the garlic organosulfide compounds and the PAPs (cap-binding proteins) and/or RTA/PAP.

A first step is selection of a garlic. There are hundreds, perhaps thousands, of distinct garlic cultivars. Most of these reproduce asexually; however, there are still in circulation today garlic cultivars that are the product of sexual production. The most tasty garlic cultivars with the most sought for health benefits are the Purple Stripes. Purple Stripes are the ancestors and antecedents of all other garlic cultivars. They are strong, complex, and richly garlicky, without being overly sulfurous. Purples Stripes need exposure to cold to grow well and develop large bulbs, though they can still produce reasonably well in some southern regions. Purples Stripes are strongly bolting hardneck cultivars. When they bolt, Purple Stripes rapidly divert their energies to the reproductive structures and away from the bulb. Bulb size suffers considerably if the scape is left uncut. Among the Purple Stripes, none is more appreciated than the Ail de Lautrec, from France. Cloves must be separated from garlic bulbs, disinfected in 25% (v/v) commercial bleach Clorox (4% sodium hypochlorite, Yuhan, Korea), and rinsed three times with sterile distilled water. The bulbs dissected from garlic cloves will be used to produce in vitro plantlets. Calli will be induced from the root segments of in vitro plantlets on MS medium supplemented with 1.0 mg/L2,4-D and 0.2 mg/L IAA for 2 months.

It is noted however, the methodology disclosed herein related to the inclusion of the PAP gene, or of the fusion protein RTA/PAP, could be used with any garlic.

A next step is selecting a means of plant transformation. Many new technologies have been developed for plant biotechnology with each a certain level of efficiency and with a specific purpose. The most commonly used is plant transformation mediated by Agrobacterium tumefaciens, a soil plant pathogenic bacterium. It allows for the introduction of foreign genes into plant cells and the subsequent regeneration of transgenic plants. It is the bacterium natural ability that transforms its host by delivering a well-defined DNA fragment, the transferred (T) DNA, of its tumor-inducing (Ti) plasmid into the host cell.

Agrobacterium-mediated transfection provides genetic transformation of many important medicinal species and has the advantage for allowing stable integration of defined DNA into the plant genome that generally results in lower copy number, fewer rearrangements and more stability of expression over generations than other transfection methods. It has been shown that it is also possible to use virus-based vectors efficiently for high transient expression of foreign proteins in transfected plants but with less efficiency. Another technique is the generation of transgenic medicinal plants by particle bombardment (direct gene transfer). This involves the use of a modified shotgun to accelerate small (1-4 μm) diameter metal particles into plant cells at a velocity sufficient to penetrate the cell wall. Another technique is electroporation. Electroporation uses brief pulses of high voltage electricity to induce the formation of transient pores in the membrane of the host cell. Those pores act as passageways through which the naked DNA can enter the host cell. And finally, chloroplast transformation to generate transgenic medicinal plants is also possible. Foreign genes are inserted into the chloroplast genome via bombardment with plasmid constructs containing a selectable antibiotic resistance marker physically linked to the gene of interest, flanked by DNA for inserting into the correct site of the chloroplast genome (Khan et al. 2009).

The GATEWAY conversion technology (Invitrogen™, Gaithersburg, Md., USA) is based on the site-specific recombination reaction mediated by phage. DNA fragments flanked by recombination sites (att) can be transferred into vectors that contain compatible recombination sites (aatB attP or attL attR) in a reaction mediated by the GATEWAY BP Clonase or LR Clonase Enzyme Mix (Invitrogen™). The entry clones, which can be considered general donor plasmids, are made by recombining the DNA fragment of interest with the flanking aatB sites into the attP site pDONR201 mediated by the GATEWAY BP CLonase Enzyme Mix. Subsequently, the fragment in the entry clone can be transferred to any destination vector that contains the attR sites by mixing both plasmids and by using the GATEWAY LR Clonase Enzyme Mix.

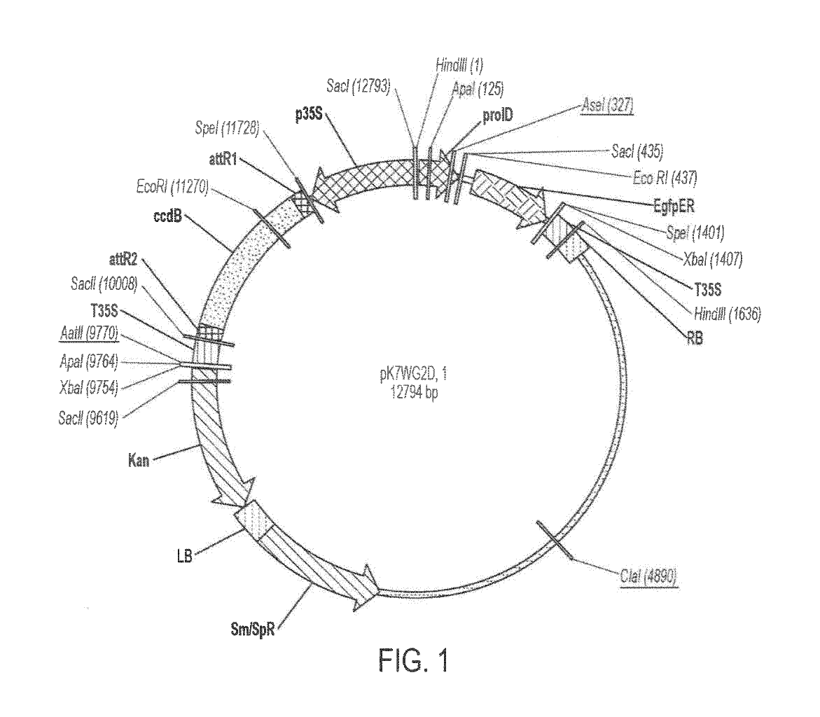

A T-DNA destination overexpression or antisense vector with an additional screenable marker, may be the selected vector. This vector is 12794 bp long, uses the promoter and terminator of the cauliflower mosaic virus (CaMV) 35S transcript, highly active in most plant cells of transgenic plants. The cassette contains the ro/D promoter fused to the coding sequences of the enhanced green-fluorescent protein (GFP) linked to the endoplasmic reticulum-targeting signal (EgfpER) and 35S terminator. The vector may also contain the native PAP and variants, isolated from Phytolacca Americana, and the hygromycin phosphotransferase (HPT) selective gene. This gene has been chosen because garlic calli are highly tolerant to the antibiotic agent kanamycin but sensitive to hygromycin. FIG. 1 illustrates the pK7WG2D vector.

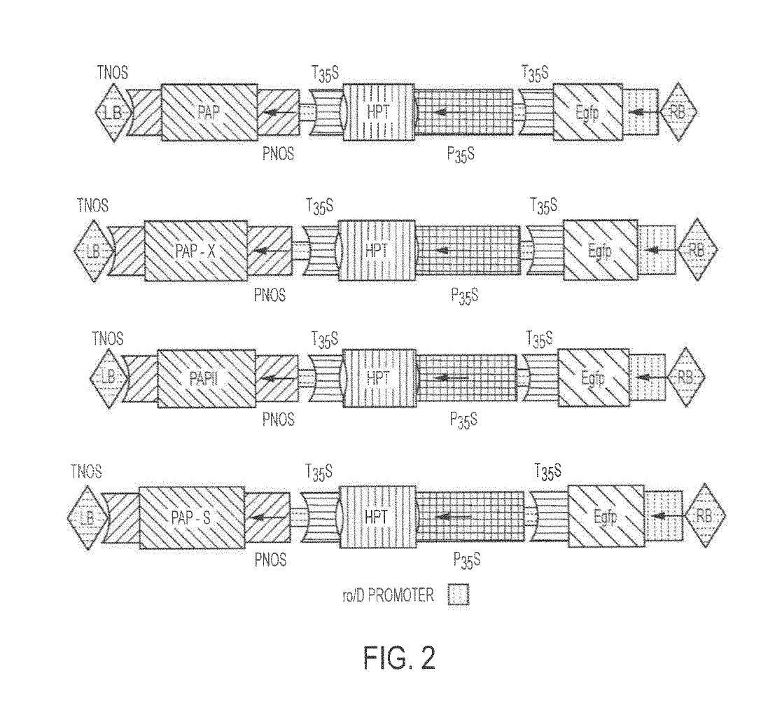

To determine which of the PAPs is the most efficient, the construction of different gene expression cassettes with variants of PAP, such as PAPII and PAP-S, is needed. These proteins are similar in molecular mass (29, 30, and 29.5 kD respectively) but are expressed at different developmental stages and in different tissues of pokeweed with different toxicity to the host. It is also possible to express truncated version of the PAP in order to increase its plant anti-viral properties, such as the PAP gene with a deletion of 36 codons from the C-terminus (PAP-X). It is noted however, that the animal antiviral properties have been shown to be lessened by such a procedure. It is also possible to change the Shine-Dalgarno sequence in the expression vector with a sequence derived from a polylinker in order to increase protein expression, by as much as two folds. This should not be necessary in the present case because transgenic tobacco plants expressing PAP or a variant have been shown to be resistant to a broad spectrum of plant viruses without showing significant toxicity. In some embodiments, PAP, or one of its variants, can be expressed in significant quantity in transgenic garlic and show significant activity against plant viruses.

Where the vectors cannot be obtained including the different PAPs, it is possible to isolate them directly from Phytolacca americana or amplified from pNT 188, a yeast vector expressing the complete unprocessed form of PAP and then modify them. The PCR product can then be cloned into the expression vector pK7WG2D to produce the pPAP-HPT-GFP vectors (as illustrated in FIG. 2).

Agrobacterium cells will be grown in LB liquid medium and mixed with chopped calli for ten minutes before being transferred onto co-cultivation media with removal of Agrobacterium cells. Following co-cultivation, Agrobacterium cells need to be thoroughly removed by several washes in sterilized water containing vancomycin, cefotaxime and hygromycin in order to only select transfected cells. A second selection based on GFP on two subcultures will also be done and actively growing calli will then be transferred to MS medium and incubated under continuous light for plant regeneration. Once this is achieved, after 4 weeks, they will be transferred to soil for further growth to maturity. As an alternative method, a particle bombardment-mediated system of garlic transformation can be developed using apical meristem-derived calli.

Two lines may be generated per vector expressing PAPs and one line with the vector without the PAP (or its variant) gene, which will be used as control to determine impact of transfection on the transgenic plant.

For genomic DNA PCR and DNA gel-blot analysis, genomic DNA must be extracted from the young leaves using cetyltrimethylammonium bromide (CTAB) method. For the RNA gel-blot analysis the RNA isolation kit (Tri Reagent, Molecular Research Center, US) can be used.

In order to test the activity of PAPs in transgenic garlic two different tests will be developed. The first one will involve an antiviral activity assay of transgenic garlic plants in order to determine the level of resistance of these transgenic plants to infection by Garlic mosaic virus, carlavirus, Garlic common latent virus and Onion yellow dwarf virus, the most common plant viruses that infect garlic. The transgenic plants of each line producing PAPs will be infected by mechanical inoculation with increasing concentrations (3 and 9 ug/ml) of those viruses. Two types of control will be used, wild type and non-expressing PAP transgenic garlic.

The second test will have a double purpose, testing the safety of eating transgenic garlic or its derived products and the added activity of PAPs to the overall effect of garlic on mice against common cold. Two types of control will be used, wild type and non-expressing PAP transgenic garlic. The common cold is associated with significant morbidity and economic consequences. On average, children have six to eight colds per year and adults have two to four despite the availability of many flu vaccines. This test will allow assessing the overall ability of transgenic garlic for common cold prevention and treatment compared to placebo, wild type garlic and non-expressing PAP transgenic garlic.

It is contemplated that PAPs and/or RTA/PAP expressing transgenic garlic will have a higher resistance to plant viruses (i.e. against mosaic viruses for example) and activity to prevent and treat a broader spectrum of infections when ingested raw or cooked (i.e. against broader range of influenza viruses vs. wild type) while being safe for human consumption. The toxicity of expressing PAPs and/or RTA/PAP is not expected to induce significant phenotype changes in transgenic garlic in any of the vectors.

### Vector

In another aspect, the invention comprises a vector comprising a polynucleotide encoding PAP or encoding a fusion protein comprising PAP. The construction of a variety of vectors having different variants of PAPs and their subsequent transfection into transgenic garlic will provide information related to expression of PAP and potential interference with garlic growth; quantity of PAP produced; effect of temperature on PAP; toxicity; effect of PAP on healing benefits of garlic; and form of PAP providing best efficiency. Different versions of the vector will allow the optimization of the properties imparted to the transgenic plant by expression of PAP. In some embodiments, the polynucleotide encodes a fusion protein comprising PAP and RTA. In various embodiments, PAP will further comprise a signal peptide, the signal peptide may comprise SEQ ID NO: 19 or SEQ ID NO: 20.

There have been contradicting studies related to the inclusion of PAP. For example, some studies show that the full-length PAP gene does not always confer significant viral resistance to the transgenic plant. This may be due to mutations after transfection. However, others have shown that mutant forms do confer high resistance to plant viruses but also affect the plant phenotype. There have been no studies however, on PAP's activity against viruses when the whole plant (or its products) is (are) used compared to when PAPs are purified to be administered intraperitoneally.

It is contemplated that by manipulating the vector to allow the transgenic plant to express novel fusion protein RTA/PAP, RTA/PAP activity may be optimized to be less cytotoxic to healthy cells and more cytotoxic to infected cells. This may further enhance selectivity toward viruses and infected cells of eukaryotic cells of the standalone fusion protein compared to PAP alone in vivo and in vitro to be used as a therapeutic. It is contemplated that RTA fusion protein to PAP will be as selective as PAP alone toward viruses and infected cells. It is contemplated that the dosage of RTA fusion protein to PAP to treat infections, pre and post, will be less than PAP alone.

In some embodiments, the N-terminal of PAP is linked to the C-terminal of RTA and in other embodiments the C-terminal of PAP is linked to the N-terminal of RTA. The arrangement of the fusion protein influences the final conformation of the mature polypeptide and ultimately influences its activity. Furthermore, the introduction of point mutations into the sequence of PAP, the signal peptide and/or RTA if present presents the opportunity to further control the activity and specificity of PAP. In some embodiments, PAP comprises an R68G mutation which reduces the RIP activity, which in some embodiments may be useful.

### Fusion Proteins

In another aspect, the invention comprises a fusion protein comprising PAP and at least one of a signal peptide and RTA. The fusion protein may be in purified or unpurified. In some embodiments, the fusion protein may be delivered as an extract or in combination with one or more pharmaceutically acceptable excipients in order to promote its delivery to a subject.

In some embodiments, the fusion protein comprises PAP and RTA. In various embodiments, PAP will further comprise a signal peptide, the signal peptide may comprise SEQ ID NO: 21 or SEQ ID NO: 22. In some embodiments, the N-terminal of PAP is linked to the C-terminal of RTA and in other embodiments the C-terminal of PAP is linked to the N-terminal of RTA. In some embodiments, PAP comprises an R68G mutation or other point mutation. In some embodiments, PAP comprises an N-terminal cysteine.

In another aspect, the invention comprises a fusion protein comprising the structure X—Y—Z, wherein X is full length RTA/PAP, Y is absent or a chemical linker and Z is a compound. In various embodiments, the compound is an antibody; a hormone; a modified hormone releasing factor; and a hormone releasing factor. The compound or linker may be linked to the N- or C-terminal of RTA/PAP or may be linked via disulfide bond.

It is thought that the transgenic plant of the present invention for enhanced resistance to pests and/or to plant viruses and improved benefit to humans upon consumption as well as the vectors fusion proteins and methods described herein will be understood from the foregoing description and it will be apparent that various changes may be made in the form, or manufacture thereof without departing from the spirit and scope of the invention or sacrificing all of its material advantages, the form hereinbefore described being merely a preferred or exemplary embodiment thereof.

### EXPERIMENTAL EXAMPLES

The invention is further described in detail by reference to the following experimental examples. These examples are provided for purposes of illustration only, and are not intended to be limiting unless otherwise specified. Thus, the invention should be in no way construed as being limited to the following examples, but rather, should be construed to encompass any and all variations which become evident as a result of the teachings provided herein.

### Example 1: Gene Cloning and Construction of Prokaryotic and Plant Expression Vectors of RICIN-A-Chain/PAP-S1 Fusion Protein and its Inhibition of Protein Synthesis

An objective of this study was to create a novel fusion protein between ricin A chain and PAP-S1 in order to achieve greater potency than PAP-S1 alone, while keeping its selectivity to infected cells and, in consequence, to reduce previously observed side effects associated to dosage. It was decided to create two versions of the fusion protein, one between ricin A chain C terminus and PAP-S1 N terminus and one between PAP-S1 C terminus and ricin A chain N terminus and determine if they were functional against both prokaryotic and eukaryotic protein synthesis apparatuses.

### Materials and Methods

The materials and methods used in Example 1 are here described.

The PureLink® Genomic Plant DNA Purification Kit, PureLink® Quick Plasmid Miniprep Kit, HisPur™ Ni-NTA Spin Purification Kit, 0.2 m, Phire Plant Direct PCR Master Mix, PureLink™ Quick Gel Extraction and PCR Purification Combo Kit, Phire Hot Start II DNA Polymerase, Gateway® BP & LR clonase II enzyme mix, Gateway® pDonr-221 vector, Gateway® pExp1-Dest vector, Expressway™ Mini Cell-Free Expression System, One Shot® TOP10 Electrocomp™ E. coli, and ElectroMAX™ A. tumefaciens LBA4404 Cells were purchased from Thermo Fisher Scientific. The pH7WG2D (Karimi et al. 2002) plant destination binary vector for A. tumefaciens mediated transformation was purchased from the university of Gent. The oligonucleotides used for PCR, creating attb PCR products and overlap extension PCR products, were synthesized by Integrated DNA Technologies. The Rabbit Reticulate Lysate TnT® Quick Coupled Transcription/Translation System and the E. coli S30 T7 High-Yield Protein Expression System were purchased from Promega.

### Amplification of DNA by PCR

### PAP-S1

Genomic DNA was prepared from seeds of Phytolacca americana using the PureLink® Genomic Plant DNA Purification Kit. Since only a partial cds of PAP-S1 (GenBank: AB071854.1) was available, different primers were designed based on the available mRNA PAP-S2 cds (GenBank: X98079.1) to sequence the complete cds of PAP-S1. The forward primer that gave the best results was SEQ ID NO: 5 5′-AGAAGCGGCAAAGGGAAGATGAA-3′ and for the reverse primer was SEQ ID NO: 6 5′-CCATTGGCCCGGCCTCTTATTT-3′. The sequence of PAP-S1 was amplified by PCR using the Phire Hot Start II DNA Polymerase kit based on the 50 ul protocol with the melting temperature of the primers determined by the TM calculator provided by Thermo Fisher Scientific. The PCR products were separated by agarose gel electrophoresis. A fragment with the expected size (about 900 bp) was extracted and used as a template for the second PCR under the same conditions. This PCR mainly amplified the fragment of about 900 bp, which was purified by agarose gel electrophoresis using the PureLink™ Quick Gel Extraction and PCR Purification Combo Kit. Below is a nucleotide blast alignment of the PAP-S1 nucleotide sequence obtained in the study described herein and the partial PAP-S1 coding sequence (cds) provided in GenBank Accession No. AB071854.1.

Phytolacca americana paps1 gene for PAP-S1, partial cds
Sequence ID: dbj|AB071854.1| Length: 783 Number of Matches: 2
Range 1: 4 to 783 GenBank Graphics ▾Next Match

| Score | Expect | Identities | Gaps | Strand |
|---|---|---|---|---|
| 1401 bits(759) | 0.0 | 773/780(99%) | 0/780(0%) | Plus/Plus |

| | | | |
|---|---|---|---|
| SEQ ID NO: 7 | | | |
| Query | 202 | AATACAATCACCTTCGACGCTGGAAATGCAACCATTAACAAGTATGCTACCTTTATGGAA | 261 |
| SEQ ID NO: 8 | | \|\|\|\|\| \|\| \|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 4 | AATACGATAACGTTCGACGCTGGAAATGCAACCATTAACAAGTATGCTACCTTTATGGAA | 63 |
| Query | 262 | TCTCTTCGTAATGAAGCGAAAGATCCAAGTTTAAAGTGTTATGGAATACCAATGTTGCCC | 321 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 64 | TCTCTTCGTAATGAAGCGAAAGATCCAAGTTTAAAGTGTTATGGAATACCAATGTTGCCC | 123 |
| Query | 322 | AATACTAATTCAACCATCAAGTACTTGTTGGTTAAGCTCCAAGGTGCAAGCCTAAAAACC | 381 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 124 | AATACTAATTCAACCATCAAGTACTTGTTGGTTAAGCTCCAAGGTGCAAGCCTAAAAACC | 183 |
| Query | 382 | ATCACACTAATGCTAAGACGAAACAACTTATATGTGATGGGCTATTCGGATCCCTACGAC | 441 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 184 | ATCACACTAATGCTAAGACGAAACAACTTATATGTGATGGGCTATTCGGATCCCTACGAC | 243 |
| Query | 442 | AATAAGTGTCGTTACCATATCTTTAATGATATTAAAGGCACTGAATACAGTGATGTGGAG | 501 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 244 | AATAAGTGTCGTTACCATATCTTTAATGATATTAAAGGCACTGAATACAGTGATGTGGAG | 303 |
| Query | 502 | AATACTCTTTGCCCAAGTTCAAATCCTCGTGTTGCAAAACCCATTAACTACAATGGCCTA | 561 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 304 | AATACTCTTTGCCCAAGTTCAAATCCTCGTGTTGCAAAACCCATTAACTACAATGGCCTA | 363 |
| Query | 562 | TATCCAACTTTGGaaaaaaaaGCAGGAGTAACCTCAAGAAATCAAGTCCAACTGGGAATT | 621 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\| | |
| Sbjct | 364 | TATCCAACTTTGGAAAAAAAAGCAGGAGTAACCTCAAGAAATCAAGTCCAACTAGGAATT | 423 |
| Query | 622 | CAAATACTCAGCAGTGACATTGGAAAAATCTCTGGACAGGGCTCGTTCACTGAAAAAATC | 681 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 424 | CAAATACTCAGCAGTGACATTGGAAAAATCTCTGGACAGGGCTCGTTCACTGAAAAAATC | 483 |
| Query | 682 | GAGGCCAAATTCCTGCTTGTAGCCATTCAAATGGTGTCAGAAGCAGCGCGATTCAAGTAC | 741 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 484 | GAGGCCAAATTCCTGCTTGTAGCCATTCAAATGGTGTCAGAAGCAGCGCGATTCAAGTAC | 543 |
| Query | 742 | ATAGAGAATCAGGTGAAGACTAATTTTAACAGAGATTTCTCCCCTAATGACAAAGTACTT | 801 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 544 | ATAGAGAATCAGGTGAAGACTAATTTTAACAGAGATTTCTCCCCTAATGACAAAGTACTT | 603 |
| Query | 802 | GACTTGGAGGAGAACTGGGGTAAGATCTCTACGGCAATTCACAATTCCAAGAATGGAGCT | 861 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 604 | GACTTGGAGGAGAACTGGGGTAAGATCTCTACGGCAATTCACAATTCCAAGAATGGAGCT | 663 |
| Query | 862 | TTACCAAAACCTCTAGAGCTAAAAAATGCAGACGGTACTAAGTGGATAGTGCTTAGAGTG | 921 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 664 | TTACCAAAACCTCTAGAGCTAAAAAATGCAGACGGTACTAAGTGGATAGTGCTTAGAGTG | 723 |
| Query | 922 | GATGAAATCAAACCTGATGTGGGACTCCTTAACTATGTTAATGGGACTTGCCAAGCAACT | 981 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\| \|\|\|\|\|\|\|\| \|\|\| | |
| Sbjct | 724 | GATGAAATCAAACCTGATGTGGGACTCCTTAACTATGTTAATGGTACCTGCCAAGCCACT | 783 |

### Ricin A Chain

The sequence of ricin A chain was isolated and amplified directly from seeds of Ricinus communis using the Phire Plant Direct PCR Master Mix. The complete sequence of ricin is widely available and the primers were designed based on the available Ricinus communis ricin gene (GenBank: X52908.1). The forward and reverse primers were selected based on the Integrated DNA Technologies primers design software to amplify ricin A chain including the linker peptide region. The forward primer selected was SEQ ID NO: 9 5′-GAGAATGCTAATGTATTTGGACAGCCA-3′ and the reverse one SEQ ID NO: 10 5′-GTATTGCGTTTCCGTTGTGGAATCT-3′. The sequence of ricin A chain with the linker peptide region was purified by agarose gel electrophoresis the same way as described for PAP-S1 (fragment of about 1 kb).

Design of Fusion DNA Sequence with Partial Attb Sequence

### PCR Extension of PAP-S1 for Fusion at the N Terminus

The N terminus of PAP-S1 was extended by PCR using the following forward primer SEQ ID NO: 11 5′-CAGTGGTACCAAATTITAATATAAATACAATCACCTTCGA-3′ (overlapping the Ricin A-chain linker peptide sequence) and reverse primer SEQ ID NO: 12 5′-AGAAAGCTGGGTAGAACATGGCATTTTGTTA-3′ using the Phire Hot Start II DNA Polymerase kit based on the 50 ul protocol. The PCR product was purified by agarose gel electrophoresis as described previously (band at 800 bp).

### PCR Extension of PAP-S1 for Fusion at the C Terminus

The C terminus of PAP-S1 was extended using the following forward primer SEQ ID NO: 13 5′-AAAAAGCAGGCTCTATAAATACAATCACCTTCGA-3′ (without the signal sequence of PAP-S1) and reverse primer SEQ ID NO: 14 5′-GTACCACTGGCCTTATAAGCAAAGAAGTTGCTTGGCAAGTC-3′ (using the Ricin linker peptide as linker between PAP-S1 C Terminus and Ricin A Chain N Terminus) using the Phire Hot Start II DNA Polymerase kit based on the 50 ul protocol. The PCR product was purified by agarose gel electrophoresis as described previously (band at 800 bp).

### PCR Extension of Ricin A-Chain at the C Terminus

The C terminus of Ricin A-chain was extended by PCR with the following forward primer SEQ ID NO: 15 5′-AAAAAGCAGGCTCTATATTCCCCAAACAATAC-3′ and reverse primer SEQ ID NO: 16 5′-TCGAAGGTGATTGTATTATATTAAATTTGGTACCACTG-3′ (overlapping PAP-S1 protein cds) using Phire Hot Start II DNA Polymerase kit based on the 50 ul protocol. The PCR product was purified by agarose gel electrophoresis (band at 850 bp).

### PCR Extension of Ricin A-Chain at the N Terminus

The N terminus of Ricin A-chain was extended by PCR with the following forward primer SEQ ID NO: 17 5′-GCTTATAAGGCCAGTGGTACCAAATITTAATATATTCCCCAAACAATAC-3′ (overlapping the ricin linker peptide) and reverse primer SEQ ID NO: 18 5′-GAAAGCTGGGTTAAAACTGTGACGATGGT-3′ using Phire Hot Start II DNA Polymerase kit based on the 50 ul protocol. The PCR product was purified by agarose gel electrophoresis (band at 850 bp).

### Fusion Protein Sequence

### At the C Terminus of Ricin

The C terminus of Ricin A-chain (including the linker peptide region) was fused to the N terminus of PAP-S1 (without the signal peptide) by overlap extension PCR. The forward and reverse primers were SEQ ID NO: 19 5′-AAAAAGCAGGCTCTATATTCCCCAAACAATAC-3′ and SEQ ID NO: 20 5′-AGAAAGCTGGGTAGAACATGGCATTTTGTTA-3′ respectively using Phire Hot Start II DNA Polymerase kit based on the 50 ul protocol. The PCR product was purified by agarose gel electrophoresis (band at 1650 bp).

### At the C Terminus of PAP-S1

The N terminus of Ricin A-chain (with the linker peptide region fused at the N terminus) was fused to the C terminus of PAP-S1 (by overlapping the linker peptide region) by overlap extension PCR. The forward and reverse primers were SEQ ID NO: 21 5′-AAAAAGCAGGCTCTATAAATACAATCACCTTCGA-3′ and SEQ ID NO: 22 5′-GAAAGCTGGGTTAAAACTGTGACGATGGT-3′ respectively using Phire Hot Start II DNA Polymerase kit based on the 50 ul protocol. The PCR product was purified by agarose gel electrophoresis (band at 1650 bp).

### DNA Sequencing

The nucleotide sequences of inserts cloned into plasmid DNA were determined using multiple primers using Sanger DNA Sequencing by an outside laboratory facility (GENEWIZ).

### Production of Recombinant PAP-S1 and Fusion Proteins

### Construction of Prokaryotic Expression Vectors

The PAP-S1 and Fusion Protein DNA sequence were each inserted into the pexp1-Dest vector using the Gateway System One Tube format from Thermo Fisher Scientific (combining the BP1 and LR reactions). The PAP-S1 and Fusion Protein DNA sequences were flanked with the attB1 and attB2 sequences (by adding them to the PCR forward and reverse primers, respectively) prior to the One Tube format.

### Construction of Plant Expression Vectors

The PAP-S1 and Fusion Protein (at the N terminus of PAP-S1) DNA sequences were each inserted into the pH7WG2D plant destination binary vector using the Gateway System One Tube format from Thermo Fisher Scientific (combining the BP and LR reactions). The signal peptide sequence of PAP-S1 was first added at the N terminus of the PAP-S1 and Fusion Protein DNA sequences by extension overlap PCR as previously described. The PAP-S1 and Fusion protein DNA sequence were then flanked with the attB1 and attB2 sequences (by adding them to the PCR forward and reverse primers, respectively) prior to the One Tube format.

### Cloning of Expression Plasmids

Both PAP-S1 and Fusion prokaryotic and plant expression plasmids were transfected by electroporation using an Eppendorf Eporator® into One Shot® TOP10 Electrocomp™ E. coli for plasmid propagation. The plasmids were then purified using the PureLink® Quick Plasmid Miniprep Kit. The plasmids were ethanol precipitated and concentrated to 500 ng/ul.

### Prokaryotic Expression

Prokaryotic expression of both PAP-S1 and Fusion proteins were achieved using the Expressway™ Mini Cell-Free Expression System. The proteins were purified using the HisPur™ Ni-NTA Spin Purification Kit, 0.2 m before being run on protein gels for confirmation.

### Inhibition of Cell-Free Protein Synthesis

The enzyme activity of the purified recombinant proteins was determined by intensity of the band on protein gel of a control against expression of the control without the recombinant proteins, after protein purification using the HisPur™ Ni-NTA Spin Purification Kit, 0.2 m, in both the Rabbit Reticulate Lysate TnT® Quick Coupled Transcription/Translation System and the E. coli S30 T7 High-Yield Protein Expression System. The control used was pEXP5-NT/CALML3 control vector as DNA template expressing an N-terminally-tagged human calmodulin-like 3 (CALML3) protein (under the T7 promoter). The concentration of CALML3 was determined for increasing concentrations of recombinant PAP-S1 and Fusion proteins by measuring band intensity on a protein gel after coomassie blue staining.

The results of the experiments are now described.

### Results and Discussion

### PAP-S1

It was difficult to extract useful genomic DNA from seeds of P. americana given the small size of its seeds and its fibrous nature. Additionally, the reverse primer led to unspecific binding, and gave unwanted PCR products. The complete cds of PAP-S1 was however determined and is now available at GenBank with the accession number KT630652. The signal peptide DNA sequence of PAP-S1 is 83% identical to the signal peptide of PAP-S2, however, the translated proteins are only 67% identical based on Blast (see below for both the nucleotide and protein alignment between the determined PAP-S1 and the PAP-S2 signal peptide.).

Sequence ID: lcl|Query_167497 Length: 72 Number of Matches: 1
Range 1: 3 to 72 Graphics

| Scores | Expect | Identities | Gaps | Strand |
|---|---|---|---|---|
| 77.0 bits(84) | 3e-20 | 60/72(83%) | 0/72(0%) | Plus/Plus |

| | | | |
|---|---|---|---|
| Query | 1 | ATGAACGTGATGCTTGTGCTTGTGGTGATGATAACATCATCGCTCATTCTTGCACCGCCT | 60 |
| SEQ ID NO: 24 | | \|\|\|\|\|\|\|\|\|\|\|\|\|\|\|\|\| \|\|\|\|\|\|\|\|\| \| \|\|\| \| \| \|\|\|\|\|\|\|\|\| \|\|\|\|\|\| \|\| | |
| Sbject | 1 | ATGAACGTGATGCTTGTAGTTGTGGTGACGTTAATAGCGTCGCTCATTGCTGCACCAACT | 60 |
| Query | 61 | TCAACTTGGGCC | 72 |
| | | \|\|\|\|\|\|\|\| \|\|\| | |
| Sbject | 61 | TCAACTTGTGCC | 72 |

Range 1: 2 to 24 Graphics

| | | | | | | |
|---|---|---|---|---|---|---|
| Scores | Expect | Method | Identities | Positives | Gaps | Strand |
| 30.4 bits(67) | 1e-08 | Compositional matrix adjust. | 16/24(67%) | 19/24(79%) | 0/24(0%) | +1 |

| | | | |
|---|---|---|---|
| SEQ ID NO: 25 | | | |
| Query | 1 | MKVMLVLVVMITSWLILAPPSTWA | 72 |
| SEQ ID NO: 26 | | MKVMLV+VV + +WLI AP ST A | |
| Sbject | 1 | MKVMLVVVVTLIAWLIAAPTSTCA | 72 |

Not to be bound by theory, it is contemplated that these differences in amino acids are significant given that signal peptide has been shown to play roles in toxicity, specificity and expression of RIPs.

### Expression Vectors and Production of Recombinant Proteins in Cell Free Expression System

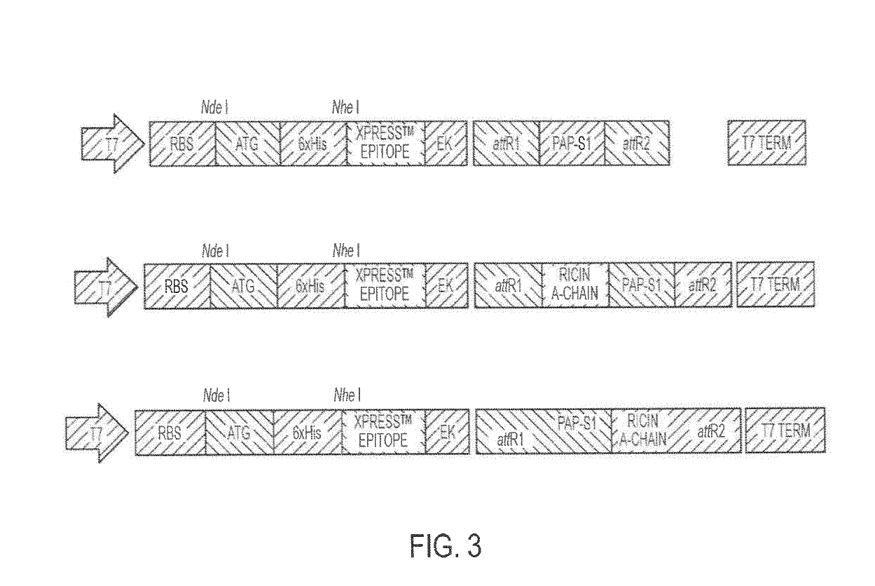

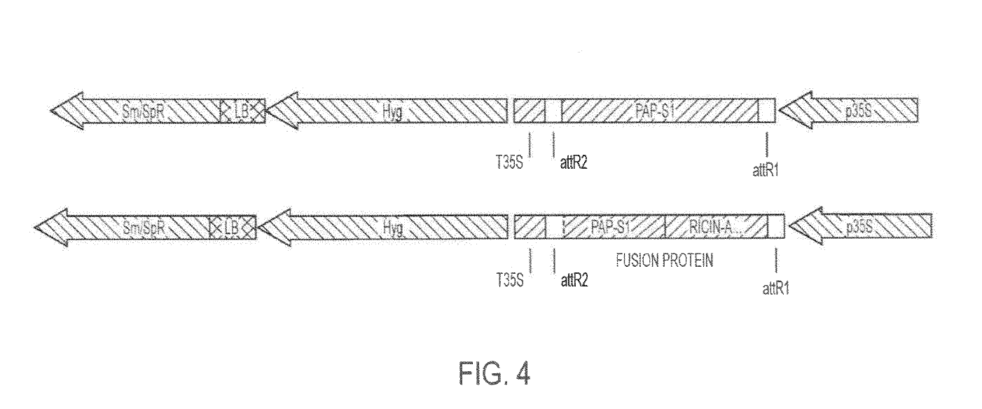

The prokaryotic and eukaryotic expression vectors were constructed following the Gateway cloning system, and no major difficulties were encountered. The DNA structures of the expression vectors used for prokaryotic expression are shown in FIG. 3 and in FIG. 4 for plant expression using binary vectors.

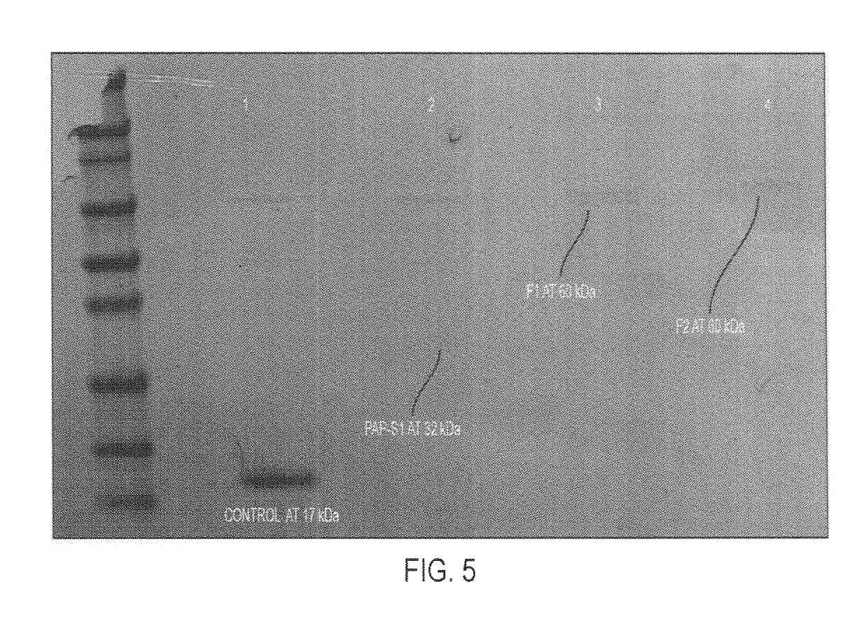

However, it was observed that it was very difficult to obtain high concentration of usable plasmids in sufficient quantities even after an overnight culture for prokaryotic expression due to the high instability of the proteins expressed. Indeed, even when the recombinant proteins were expressed in minimal amounts due to the leaking nature of the vector, it was found that they were extremely toxic to E. coli as was previously observed. For this reason, PCR amplifications of the genes of interests from the T7 promoter to the T7 terminator regions were done from the plasmids. Linear DNA (including the control) was used in the Expressway Mini Cell free System to generate the recombinant proteins. The proteins were expressed and visible bands showed up where expected on protein gel stained with coomassie blue (FIG. 5), but it is important to note that the levels of expression for the three recombinant proteins were very low compared to the control protein and, more importantly, the level of expression of the fusion protein at the PAP-S1 C terminus (F1) was higher than that of the fusion protein at PAP-S1 N terminus (F2). The expression levels of PAP-S1 were the lowest and barely measurable. The low levels of expression of the recombinant proteins were anticipated since the recombinant proteins were expected to be toxic to prokaryotic ribosomes. PAP-S1 is highly toxic to prokaryotic ribosomes as it can cleave not only adenine, but also guanine from the rRNA of Escherichia coli as above mentioned. In regard to F1 and F2, the recombinant proteins were successfully produced and their low expression levels show that they are both functional.

### Activity of Recombinant F1, F2 and PAP-S1

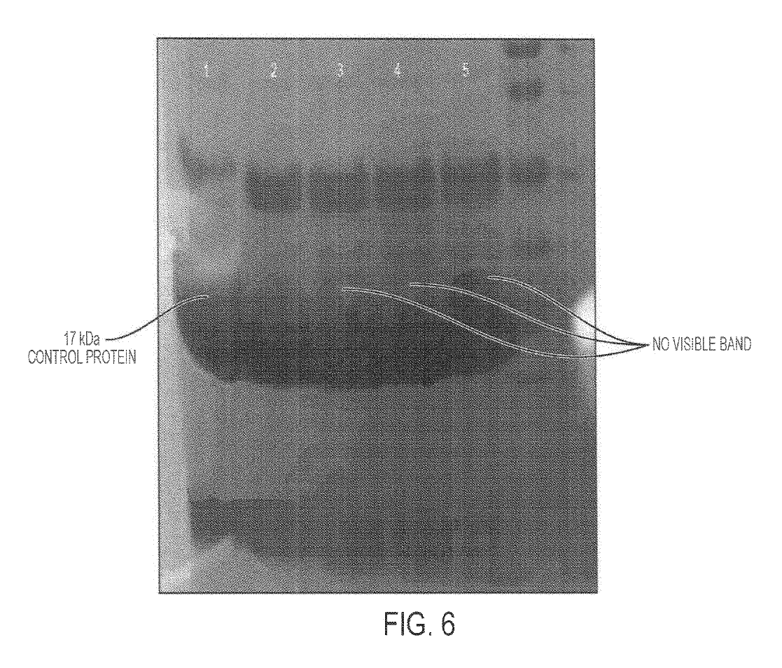

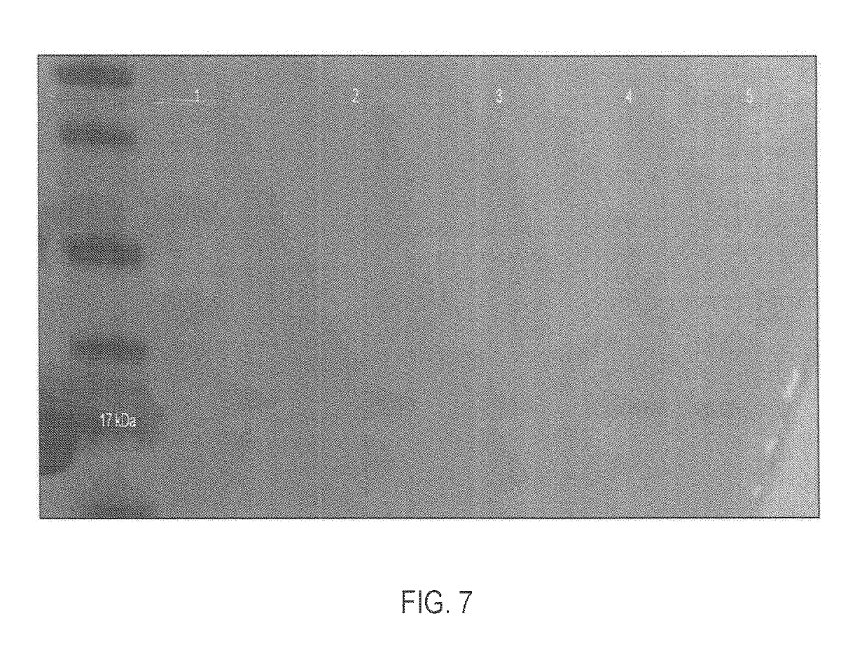

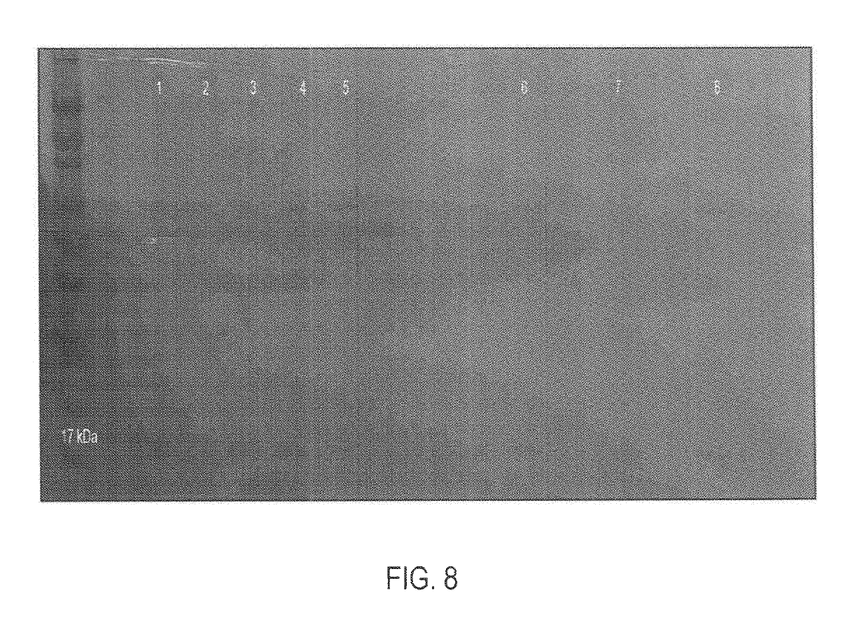

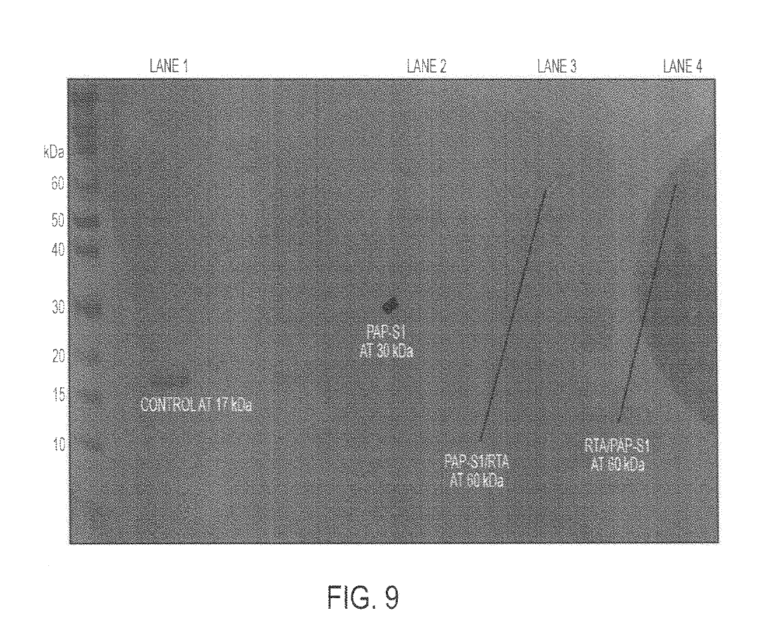

In order to have a better idea of the activity of the recombinant proteins, protein inhibition synthesis assays in both prokaryotic and eukaryotic systems were achieved using various kits. The kit Rabbit Reticulate Lysate TnT® Quick Coupled Transcription/Translation System was used to determine activity in a eukaryotic system where the same volume of PAP-S1, F1 and F2 was used (18 μl of each His-Tag purified proteins). It is important to note that the expression of F1 was shown to be greater than both F2 and PAP-S1 in the cell free expression system. Thus, 36% volume of F1 would be more concentrated than 36% F2 and than 36% PAP-S1. As shown in FIG. 6, the control gave a blur at the expected size (the blur is due to globins) while all the others gave no activity whatsoever. This was expected since PAP-S1, F1 and F2 are supposed to be extremely potent in a eukaryotic system (in the order of 1 nM for total inhibition). The same volume of PAP-S1, F1 and F2 were then used in two different prokaryotic systems, the E. coli S30 T7 High-Yield Protein Expression System and the Expressway™ Mini Cell-Free Expression System. Similar results were observed where low volume of PAP-S, F1 and F2 showed some protein inhibition (Lanes 3, 4 and 5 in FIGS. 8 and 9). In order to determine which of F or F2 is the most active one, increasing volume of F1 and F2 were used against control DNA. It was observed that F2 is more active than F1 with increasing concentrations, and even higher than PAP-S1. FIG. 8 shows 25% volume of F2 to be as potent as 36% volume of F1, and 36% volume of F2 almost completely inhibits protein synthesis while F1 at 36% still shows a visible band at 17 kDa. This difference in activity might be due to the C terminus of Pap-S1 being free in F2 as it was previously observed that the C terminus has distinct roles in transport to the cytosol, ribosome depurination and cytotoxicity (Baykal et al. 2007). It could also be due to a conformation difference between F1 and F2.

### Conclusion

The fusion protein between Ricin A chain C terminus and PAP-S1 N terminus was observed to be functional and active in both eukaryotic and prokaryotic cell free system with a visible increase in activity compared to the fusion protein between Ricin A chain N terminus and PAP-S1 C terminus under the same conditions. It was also observed that it was higher in activity than PAP-S1 in a prokaryotic system and at least identical in a eukaryotic system. The expression vectors for Plant expression were thus built based on the F2 version and were found to be extremely stable. The expression vectors for the prokaryotic systems were found to be extremely unstable, and thus, a better expression vector must be developed if E. coli expression is sought after. There are alternative expression systems to E. coli available now commercially and easily accessible. Additional research should be done in order to determine accurately activity of fusion protein F2 compared to PAP-S1 as it could be a viable more potent and less cytotoxic alternative to PAP-S1 alone for both agricultural and therapeutic applications.

### Example 2: Expression of Pokeweed Antiviral Protein Isoform S1 (PAP-S1) and of Ricin-A-Chain/PAP-S1 Novel Fusion Protein (RTA/PAP-S1) in Escherichia coli and their Comparative Inhibition of Protein Synthesis In Vitro

### Materials and Methods

The materials and methods used in Example 2 are here described.

The PureLink® Genomic Plant DNA Purification Kit, HisPur™ Ni-NTA Spin Purification Kit, 0.2 m, Phire Plant Direct PCR Master Mix, PureLink™ Quick Gel Extraction and PCR Purification Combo Kit, Phire Hot Start II DNA Polymerase,

Expressway™ Mini Cell-Free Expression System and NuPAGE™ 10% Bis-Tris Protein Gels, 1.5 mm, 15-well were purchased from Thermo Fisher Scientific. The oligonucleotides used for PCR and overlap extension PCR products, were synthesized by Integrated DNA Technologies. The Rabbit Reticulate Lysate TnT®

Quick Coupled Transcription/Translation System, Luciferase Assay System and the E. coli S30 T7 High-Yield Protein Expression System were purchased from Promega. The cDNA coding for the PAPS1[R68G] protein and Ricin-A-Chain/PAP-S1R68G were chemically synthesized with optimization for E. coli expression by GenScript. The E. coli pT7 expression vector and E. coli strains BL21(DE3) were purchased from Proteogenix. The total sample protein contents was analyzed using Qubit™ 3.0 Fluorometer and Bradford protein assay, the luciferase assay readings were achieved using a Perkin Elmer EnVison Microplate Reader and all the gels were analyzed using GelAnalyzer 2010.

E. coli Cell Free Expression and E. coli Protein Synthesis Inhibition
Design of the DNA Sequences of the Proteins for E. coli Cell Free Expression System

The DNA sequences were isolated and designed as explained above. In short, fresh seeds of both Ricinus communis and Phytolacca Americana were purchased and Ricin A Chain (RTA) and PAP-S1 (with native signal peptide) isolated and PCR amplified. The 6-His tag was added to PAP-S1 at the C terminal. The RTA/PAP-S1 fusion protein was achieved through PCR extension, using the native RTA polylinker, between RTA C terminal and PAP-S1 (without the signal peptide) N terminal with the 6-His tag at RTA N terminal. The PAP-S1/RTA fusion protein was achieved through the same means but between PAP-S1 C terminal and RTA N terminal with the 6-His tag at PAP-S1 N terminal.

E. coli Cell Free Expression and Purification

The PAP-S1, PAP-S1/RTA and RTA/PAP-S1 were produced using Expressway™ Mini Cell-Free Expression System as described above. In short, Linear DNA was used for all proteins in thrice the volume (150 uL) for PAP-S1 and PAP-S1/RTA and twice the volume for RTA/PAP-S1 using the T7 promoter. System. The proteins were purified using the HisPur™ Ni-NTA Spin Purification Kit, 0.2 m before being run on protein gels for confirmation. The total protein content was determined using Qubit™ 3.0 Fluorometer and the gels analyzed with GelAnalyzer2010.

Inhibition of Protein Synthesis in E. coli System

The enzyme activity of the purified recombinant proteins was determined by intensity of the band on protein gel of a control against expression of the control without the recombinant proteins, after protein purification using the HisPur™ Ni-NTA Spin Purification Kit, 0.2 m, as described above using the E. coli S30 T7 High-Yield Protein Expression System. The control used was pEXP5-NT/CALML3 control vector as DNA template expressing an N-terminally-tagged human calmodulin-like 3 (CALML3) protein (under the T7 promoter). The concentration of CALML3 was determined for increasing concentrations of recombinant PAP-S1 and fusion proteins by measuring band intensity on a protein gel after Coomassie blue staining by GelAnalyzer 2010.

E. coli In Vivo Expression System and Rabbit Reticulate Lysate Protein Synthesis Inhibition
Design of the DNA Sequences of the Proteins for E. coli In Vivo Expression System

The cDNA coding for a mutated version of PAP-S1 and RTA/PAP-S1, namely PAP-S1R68G and RTA/PAP-S1R68G, were chemically synthesized with optimization for E. coli expression by GenScript. The mutated form of PAP-S1 was used in order to reduce E. coli ribosomes depurination by PAP-S1 and RTA/PAP-S1 while safeguarding their Eukaryotic ribosome depurination activities. The native PAP-S1 signal peptide was kept for PAP-S1R68G with the addition of an E. coli signal peptide of two amino acids at PAP-S1R68G and RTA/PAP-S1R68G N terminal and with the 6-His tag at PAP-S1R68G and RTA/PAP-S1R68G C terminal.

E. coli In Vivo Expression and Purification

### Expression Vector

The cDNA sequences described above were cloned in an E. coli pT7 expression vector using the NcoI/XhoL1 cloning strategy.

### In Vivo Production of Proteins and Purification

### Production

Optimal conditions for E. coli B121(DE3) were determined for PAP-S1R68G and RTA/PAP-S1R68G respectively in small volumes before being scaled up to IL production culture. In short, bacteria starter were obtained by incubation at 37° C. and then followed by IPTG induction at specific temperatures and incubation times. The bacteria were then harvested by centrifugation, followed by Lysis. The supernatant was collected alter centrifugation for both proteins; the native proteins extracts.

### Purification

The purification of the native proteins extracts was achieved by affinity versus His-tag on Ni-resin. The equilibration was done with a standard binding buffer. The wash and elution by imidazole shift. After purification, fractions of interest were pooled and concentrated and analyzed by SDS-PAGE. The final concentration was determined by Bradford protein assay.

### Rabbit Reticulate Lysate Protein Synthesis Inhibition

The inhibition activity of PAP-S1[R68G] and RTA-PAP-S[R68G] were tested by using the Rabbit Reticulate Lysate TnT® Quick Coupled Transcription/Translation System and the Luciferase Assay System. Briefly, each transcription/translation reaction run was performed according to the instructions for use (IFU) in the presence of a T7 Luciferase reporter DNA, and the Luciferase expression level was determined with a Perkin Elmer EnVison Microplate Reader. Transcription/translation runs were done twice with and without addition of five different concentrations of PAP-S1R68G and RTA-PAP-S1R68G in order to determine the inhibitory effect of the proteins. PAP-S1R68G and RTA-PAP-S1R68G concentrations were adjusted by taking samples purity into consideration.

### Results and Discussion

### Production and Purification of Recombinant Proteins in Cell Free Expression System

The proteins were expressed and visible bands showed up where expected on protein gels stained with Coomassie blue (FIG. 9), but it is important to note that the levels of expression for the three recombinant proteins were very low compared to the control protein and, more importantly, with a much lower purity. The expression levels of PAP-S1 were the lowest. The low levels of expression of the recombinant proteins were expected since the proteins are toxic to prokaryotic ribosomes. The total protein content was determined using Qubit™ 3.0 Fluorometer for each sample and the purity of the sample using GelAnalyzer 2010 and are presented in Table 1.

TABLE 1

| | Total | Reaction | | Final Product |
|---|---|---|---|---|
| Protein | Produced | Volume | Purity | in 50 μL |
| CalmL3 - | 5338 ng | 50 μL | 68% | 106.8 ng/μL |
| control | | | | |
| PAP-S1 | 4514 ng | 150 μL | 25.90% | 90.3 ng/μL |
| PAP-S1/RTA | 5924 ng | 150 μL | 33.60% | 118.5 ng/μL |
| RTA/PAP-S1 | 3626 ng | 100 μL | 29.40% | 72.5 ng/μL |

Activity of Recombinant Proteins in E. coli Protein Synthesis

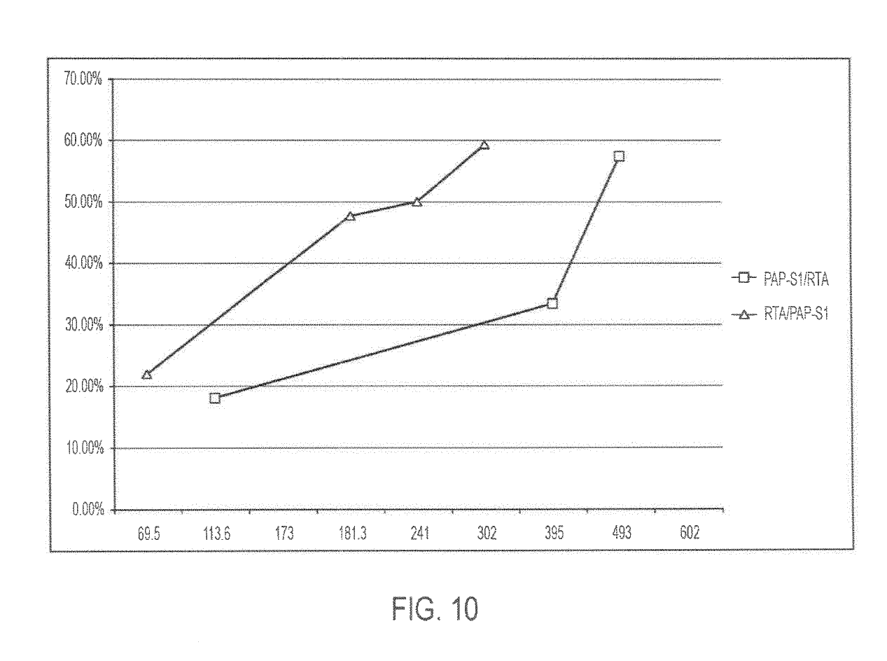

In order to determine which fusion protein was the most active in inhibiting E. coli protein synthesis, two different prokaryotic kits were used to rule out variation from one kit to another, the E. coli S30 T7 High-Yield Protein Expression System and the Expressway™ Mini Cell-Free Expression System. The same control protein was produced, namely CalmL3. Various concentration of recombinant proteins were added to each reaction and the amount of CalmL3 protein produced was compared to the amount of CalmL3 produced without the addition of any recombinant protein and plotted as percent protein inhibition compared to control versus concentration (FIG. 10). The IC₅₀ of PAP-S1/RTA was found to be of around 460 nM and of RTA/PAP-S1 of around 241 nM while the one of PAP-S1 is known to be around 280 nM. Those initial results confirm RTA/PAP-S1 as more potent than PAP-S1/RTA as it was expected since the C terminal was observed to play a role in activity and also probably due to a difference in conformation. Those results also confirm that RTA/PAP-S1 is more potent than PAP-S1 alone. The increased in activity of RTA/PAP-S1 compared to PAP-S1 alone can probably be explained by the fact that PAP-S1 and RTA do not dock onto the ribosome at the same site. Indeed, it was found that after PAP-S1 partially depurinates E. coli ribosome, RTA is able to depurinate the same ribosome while RTA cannot depurinate an intact E. coli ribosome on its own.

Production and Purification of Recombinant Proteins in E. coli Culture

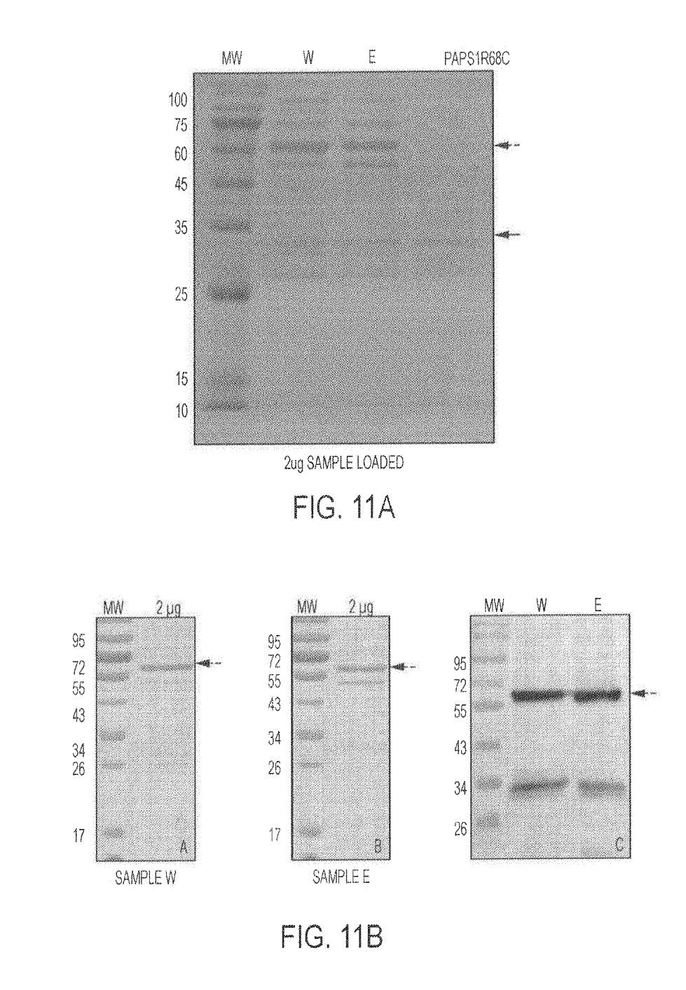

Due to problems producing the wild type proteins in both insect and yeast cell culture, a mutated form of PAP-S1 (PAP-S1R68G) with native signal peptide, believed to greatly reduce activity in E. coli and somewhat reduce activity in eukaryotic cells in E. coli cell culture was produced. PAP-S1R68G was produced in its native environment with a purity of 60% and RTA/PAP-S1R68G was produced in its native environment with a purity of 55% as shown in FIG. 11A (purity determined by GelAnalyser 2010). More than 400 μg of each protein was produced. It was observed, however, that the 6-His tag purification process was very inefficient and a lot of protein was lost in the wash, which was kept for further analysis, as shown in FIG. 11B. This is probably due to the 6-His tag being hidden by the protein in its final conformation. The double band is probably due to protein degradation or unwanted recombination at the production site by E. coli cells.

The final concentration was determined using a Bradford protein assay for each sample and the purity of the sample using GelAnalyzer 2010 and are presented in Table 2. The yield was very low, but again, a lot of protein was lost in the washes as the 6-His tag purification does not appear to be the right system for those proteins.

TABLE 2

| | Final | | | |
|---|---|---|---|---|
| Native protein | concentration | Volume | Final product | Purity |
| PAP-S1R68G | 0.23 mg/ml | 3.2 ml | 0.736 mg | 60% |
| RTA/PAP- | 0.53 mg/ml | 1.35 ml | 0.716 | 55% |
| S1R68G | | | | |
| (sample E) | | | | |
| RTA/PAP- | 0.26 mg/ml | 4.8 ml | 1.25 mg | 65% |
| S1R68G | | | | |
| (sample W) | | | | |

### Activity of Recombinant Proteins in Rabbit Reticulate Lysate TnT® System

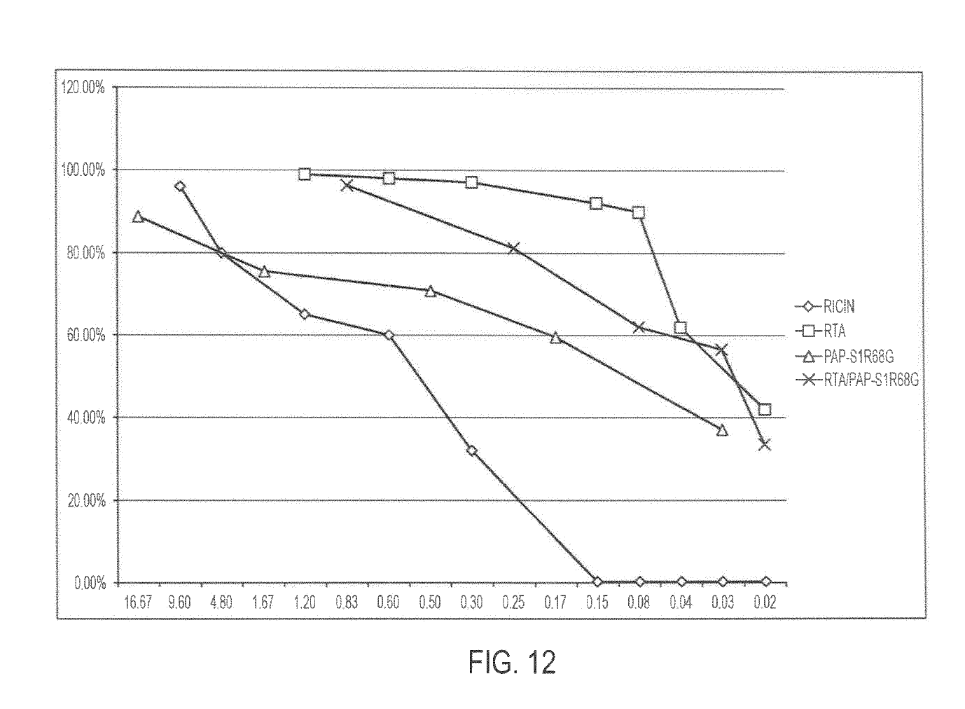

The inhibition activity of PAP-S1R68G and RTA/PAP-S1R68G were determined using 5 different concentrations of PAP-S1R68G and RTA/PAP-S1R68G on the Rabbit Reticulate Lysate TnT® system using Luciferase as control and then a Luciferase assay was used to determine Luciferase expression level using a luminometer. The comparative plot is shown in FIG. 12 and includes previous data on Ricin and RTA obtained similarly. As can be observed, RTA/PAP-S1R68G behaves more like RTA than PAP-SIR68G and has an IC₅₀ at 0.025 nM (similar to RTA 0.03 nM) against 0.06 nM for PAP-SIR68G. The total inhibition is attained at 0.83 nM for RTA/PAP-S1R68G while PAP-SIR68G barely reaches 90% at 16.67 nM, probably due to the single point mutation (R68G). It is also interesting to note that PAP-S1R68G has about the same IC₅₀ as PAP-S2 (0.07) but a much higher total inhibition point (around 1.2 nM for PAP-S2). These results not only show that RTA/PAP-S1R68G is at least twice faster than PAP-S1R58G but also 16 times more potent. It is actually comparable to RTA and can thus be assumed that non-muted RTA/PAP-S1 is going to be even faster.

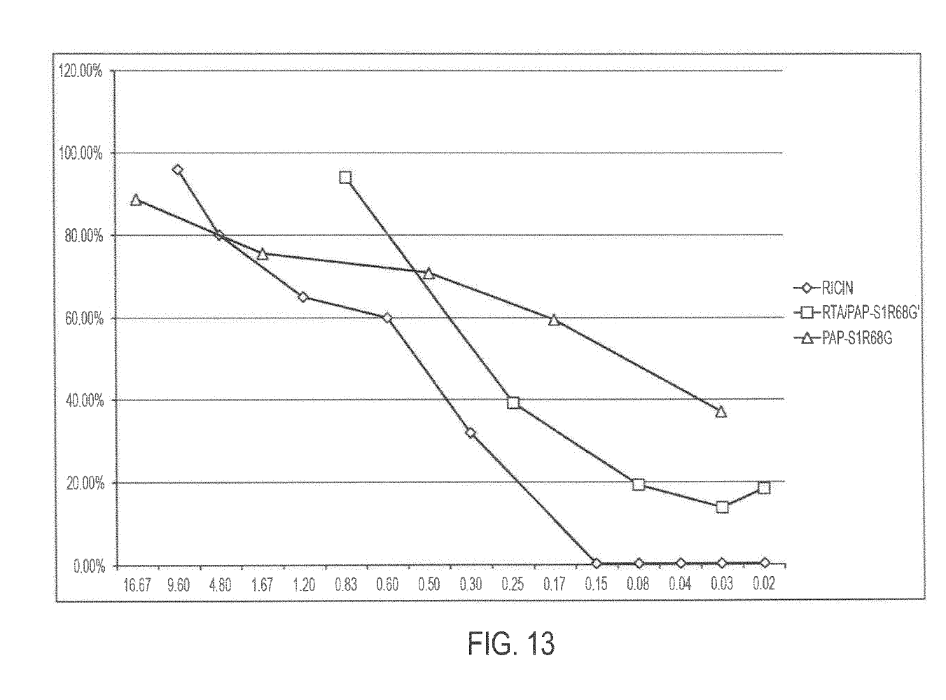

A point of great interest however, during the course of the experiments the Applicant realized that they had more protein in the wash than in the elution. It was assumed that it was due to the 6-His tag being hidden by the final conformation of the protein. However, to make sure it was just a binding issue and not a different protein altogether, bioactivity tests were run under the same conditions and the results are shown in FIG. 13. The results are depicting a completely different behavior that is more like wild type Ricin protein, but with an IC₅₀ of 0.3 nM against 0.5 nM for wild type Ricin.

The existence of two conformations of the same protein was to be expected as this fusion protein is new and it is difficult to predict exact conformation. To make sure there was no mutation at the plasmid level, cDNA isolated from a single colony was sequenced and no mutations were observed. A prediction software showed high probability of existence of a di-sulfide bond between C173 and C539. This could explain the observed behavior and the difficulty in using 6-His tag for affinity purification. However, this remains to be proven, as this behavior was not observed in cell free expression system and using an antibody against PAP-S1 might solve the yield issue and any interaction with the 6-His tag itself.

### CONCLUSION

The fusion protein between Ricin A chain C terminus and PAP-S1 N terminus was observed to be functional and active in both eukaryotic and prokaryotic cell free system with a great increase in both speed and potency compared to PAP-S1 alone. It was also observed that it was comparable in activity to Ricin A chain in eukaryotic system. It is the opinion of the authors that additional research should be done in order to determine both cytotoxicity and selectivity of RTA/PAP-S1 to PAP-S1 against a wide range of mammalian infectious diseases including some type of cancers and wide range of plant infectious diseases as it would be a much more viable, potent and less cytotoxic alternative to PAP-S1 alone for both agricultural and therapeutic applications.

The contents of each patent or application as well as each publication cited, explicitly including information and sequences associated with Genbank Accession Nos., herein are hereby incorporated by reference in their entirety.

1. A transgenic plant expressing Pokeweed Antiviral Protein (PAP) or a fusion protein comprising PAP.

2. The transgenic plant according to claim 1 wherein PAP has 95% or greater sequence identity to SEQ ID) NO: 2.

3. (canceled)

4. The transgenic plant according to claim 1, wherein the resistance of the transgenic plant to plant viruses is enhanced relative to comparable wild type plants.

5. The transgenic plant according to claim 1, wherein the transgenic garlic plant has improved antiviral activity when consumed by an animal compared to the antiviral activity of a wild type plant when consumed by an animal.

6. The transgenic plant of claim 5 wherein the animal is a human.

7. The transgenic plant according to claim 1 wherein the expression of PAP or fusion protein comprising PAP does not interfere with plant growth as compared to wild type.

8. The transgenic plant according to claim 1 wherein PAP or the fusion protein comprising PAP further comprises a signal peptide.

9. The transgenic plant according to claim 8 wherein the signal peptide comprises SEQ ID NO: 25 or SEQ ID NO: 26.

10. The transgenic plant according to claim 1 wherein the transgenic plant expresses a fusion protein comprising PAP and ricin A-chain (RTA).

11. The transgenic plant according to claim 10 wherein the N-terminal of PAP is linked to the C-terminal of RTA.

12. The transgenic plant according claim 10 wherein the C-terminal of PAP is linked to the N-terminal of RTA.

13. The transgenic plant according to claim 10, wherein RTA has 95% or greater sequence identity to SEQ ID NO: 4.

14. The transgenic plant according to claim 10, wherein RTA has 98% or greater sequence identity to SEQ ID NO: 4.

15. The transgenic plant according to claim 1 wherein the plant is a member of genus Allium.

16. The transgenic plant according to claim 1 wherein the plant is Allium sativum.

17. The transgenic plant according to claim 1 wherein the transgenic plant is a medicinal plant.

18. The transgenic plant according to claim 1 wherein the transgenic plant is a garlic plant

19. The transgenic garlic plant according to claim 18 wherein the transgenic garlic plant is a purple stripe cultivar.

20. A vector comprising a polynucleotide encoding PAP or encoding a fusion protein between a type II RIP and PAP.

21. The vector according to claim 20 wherein PAP has 95% or greater sequence identity to SEQ ID NO: 1.

22. (canceled)

23. The vector according to any one of claims 20-22 wherein the type II RIP is RTA.

24. The vector according to claim 23 comprising a fusion protein wherein RTA has 95% or greater sequence identity to SEQ ID NO: 3.

25. (canceled)

26. The vector according to claim 23 wherein the N-terminal of PAP is linked to the C-terminal of RTA.

27. The vector according claim 23 wherein the C-terminal of PAP is linked to the N-terminal of RTA.

28. The vector according to claim 20 wherein PAP or the fusion protein comprising PAP further comprises a signal peptide.

29. The vector according to claim 28 wherein the signal peptide comprises SEQ ID NO: 23 or SEQ ID NO: 24.

30. The vector according to claim 20, wherein the polynucleotide is operably linked to a promoter.

31. The vector according to claim 20, wherein PAP or the fusion protein comprising PAP comprises the point mutation R68G.

32. The vector according to claim 20, wherein PAP or the fusion protein comprising PAP comprises an N-terminal cysteine.

33. A method of producing a transgenic plant comprising transforming a parent plant with a vector according to claim 20.

34. A fusion protein comprising PAP and at least one of a signal peptide and a type II RIP.

35. A fusion protein according to claim 34 wherein PAP has 95% sequence identity to SEQ ID NO: 2.

36. A fusion protein according to claim 34 wherein PAP has 98% sequence identity to SEQ ID NO: 2.

37. The fusion protein according to claim 34 wherein PAP comprises an N-terminal cysteine.

38. The fusion protein according to claim 34 comprising RTA.

39. A fusion protein according to claim 38 wherein the fusion protein comprises RTA and RTA has 95% or greater sequence identity to SEQ ID NO: 4.

40. A fusion protein according to claim 38 wherein the fusion protein comprises RTA and RTA has 98% or greater sequence identity to SEQ ID NO: 4.

41. The fusion protein according to claim 38 wherein the N-terminal of PA P is linked to the C-terminal of RTA.

42. The fusion protein according to claim 38 wherein the C-terminal of PAP is linked to the N-terminal of RTA.

43. The fusion protein according to claim 40 further comprising a signal peptide.

44. The fusion protein according to claim 43 wherein the signal peptide comprises SEQ ID NO: 25 or SEQ ID NO: 26.

45. A fusion protein comprising the structure X—Y—Z, wherein X is full length PAP or a fusion protein comprising full length RTA and full length PAP, Y is absent or a chemical linker and Z is a compound.

46. The conjugate according to claim 45 wherein the compound comprises one or more of: an antibody; a hormone; a modified hormone releasing factor; and a hormone releasing factor.

47. The conjugate according to claim 45 or claim 46 wherein Y is a chemical linker.

48. The conjugate according to claim 48 wherein the chemical linker is a polylinker.

49. The conjugate according to any one of claims 48-51 wherein the compound is linked to the N-terminal of PAP or a fusion protein comprising full length RTA and full length PAP.

50. The conjugate according to claim 48 wherein the compound is linked to the C-terminal of PAP or a fusion protein comprising full length RTA and full length PAP.

51. The conjugate according to claim 48, wherein PAP contains an R68G point mutation.
